(19) United States
(12) Patent Application Publication
(10) Pub. No.: US 2007/0015292 A1
(43) Pub. Date: Jan. 18, 2007

(54) Apparatus, System and Method for Evaluating Fluid Systems

(76) Inventors: Barry Ruddick, Halifax (CA); Christopher Thomas Taggart, Upper Sackville (CA)

Correspondence Address: HERMAN & MILLMAN, 425 UNIVERSITY AVENUE, SUITE 300, TORONTO, ON, M5G 1T6, CA

(21) Appl. No.: 11/458,287

(22) Filed: Jul. 18, 2006

Related U.S. Application Data
(60) Provisional application No. 60/699,845, filed on Jul. 18, 2005.

(51) Int. Cl. G01N 33/551; C12M 1/34; B03C 1/02; B01D 35/06

(52) U.S. Cl. 436/524; 435/287.2; 210/222

(57) ABSTRACT

In one aspect, the invention relates to a system for evaluating a fluid system, which includes one or more batches of magnetically-responsive particles and one or more magnetic particle-collectors. In some embodiments, the magnetically-responsive particles have a composition that includes elements that are attracted by magnetic fields, but are not themselves magnetically-attractive. Each batch of magnetically-responsive particles can be released into the fluid system at a location upstream from the magnetic particle-collectors. The particles will follow fluid paths, experiencing effects, such as for example and without limitation, dispersion, diffusion and advection as they move through the fluid system. As particles reach the vicinity of the magnetic particle-collectors, the magnetically-responsive particles will be attracted into and held by the magnetic field and physical structure of the magnetic particle-collectors. The magnetically-responsive particles collected by each collector are then quantified and compared to the number of magnetically-responsive particles collected by other collectors, thereby providing a means for collecting information about the fluid system.

### FIELD OF THE INVENTION

The invention relates to collecting information about fluid systems, and, in particular to apparatus, systems and methods for evaluating fluid systems.

### BACKGROUND OF THE INVENTION

When evaluating various situations in which fluid flow is an issue (such situations are generally referred to as fluid systems hereinafter) corresponding numerical flow models are often employed to gain insights and make decisions. However, a fluid system is often very dynamic and collecting data to create such models and/or make inferences about phenomena occurring within a particular fluid system is challenging. In particular, it is often quite challenging to accurately measure, across a range of scales (e.g. distances, areas, volumes and time), the motion of a fluid (e.g. sea-water or a mixed-phase sewage slurry) and the dispersion, mixing and/or advection of suspended biological propagules (e.g. eggs, larvae, etc.), particulates, pollutants, toxins, natural and manmade dyes and sediments in the fluid. Consequently, many numerical flow models (e.g. used for constrained fluid flows, rivers, lakes, coastal and open ocean systems) are flawed for lack of validation or hindered in development due to limited empirical data.

Conventional techniques and technologies for collecting data to evaluate a fluid system include instrument-carrying and/or drogued drifters, fixed current meters, dyes and chemical tracers. Employing these techniques and technologies is often prohibitively expensive in terms of equipment and vessel or platform operating costs and the number of man-hours required to collect the data. Instrument-carrying and/or drogued drifters are relatively expensive and often require careful monitoring to ensure that they are not lost and/or damaged during use. Dyes and other chemical tracers are often absorbed, adsorbed, degraded and/or become diluted to the point where they are below the reliable measurement sensitivities of instruments employed to detect them. Further, most, if not all, conventional technologies and techniques provide information at time and space scales too large to provide accurate sub-grid estimates of dispersion, a poorly understood quantity that must be accurately parameterized if numerical models are to work well. Moreover, specific variants of apparatus, systems and methods developed and/or adapted for environmental field applications are not always suitable for industrial applications, and vice versa.

Specifically, in many environmental field applications, such as in aquatic and wetland areas, those studying aspects of the environment, are not able to accurately collect the information they are looking for using conventional techniques and technologies. Dyes and other chemical tracers must meet environmental protection standards and only be released in amounts small enough to ensure that build-up in and/or damage to the environment does not occur. Subsequently, the use of dyes and chemical tracers provides only limited and often flawed results. On the other hand, drifters are typically too large to move through wetland areas and closely follow suspended sediments, biological propagules (e.g. eggs, larvae, etc.) and the like, to accurately collect the type of data sought. Drifters are also typically too expensive to afford losing in open water, in wetlands, or during inclement weather and are incapable of tracing fluid flow sediments on/near bottom.

Industrial applications often have similar and/or analogous problems to those experienced in environmental field applications. Additionally, in many industrial applications a fluid system also includes aggressive chemicals and/or debris that can contaminate and/or damage drifters, degrade, adsorb, absorb and dilute dyes and chemical tracers, and damage instruments used to detect dyes and chemical tracers. Moreover, in constrained fluid flows, such as for example sewers, storm drainage systems and industrial pipelines, down-hole pipes (and piping) drifters of any type are an exceptionally poor option as they can clog flow channels and alter fluid flows simply by being present in the fluid system.

### SUMMARY OF THE INVENTION

According to an aspect of an embodiment of the invention there is provided a magnetically-responsive particle that is attracted to a magnet, for use in a fluid with a first density comprising: a base material with a second density that is lower than the first density of the fluid; and a magnetically-responsive material with a third density that is higher than the density of the first density of the fluid; wherein the base material and the magnetically-responsive material are combined in a ratio that establishes a fourth density for the magnetically-responsive particle that is set relative to the first density.

According to an aspect of an embodiment of the invention there is provided a magnetic particle-collector (defined as a collector with one or more magnetic element(s) that collects magnetically-responsive particles) comprising: a body defining a fluid flow path; and at least one magnetic element, free-standing, attached, suspended with, or within or integrated into the body. In some embodiments, the magnetic particle-collector includes a self-vaning mechanism and/or a flow-control device for control the velocity of a fluid within the body.

According to an aspect of an embodiment of the invention there is provided a kit of parts for evaluating a fluid system, comprising: a batch of magnetically-responsive particles; and at least one magnetic particle-collector.

According to an aspect of an embodiment of the invention there is provided a method of manufacturing magnetically-responsive particles comprising: combining a first amount of a base material with a second amount of a magnetically-responsive material to create a magnetically-responsive composition, wherein the first and second amounts are determined from a mass ratio derived from the target density of the composition; and forming the magnetically-responsive particles. A third material may be used to bind the first and second materials. A fourth material may be used to coat and/or strengthen the material and/or the magnetically-responsive particle

According to an aspect of an embodiment of the invention there is provided a batch of magnetically-responsive particles according to claim 1, the batch comprising a mixture of particles of different fourth densities that are a combination of negatively buoyant, neutrally buoyant, and positively buoyant in the fluid. In some embodiments, the batch further comprises a mixture of magnetically-responsive particles of different sizes, shapes, identifying colours or dyes, specific gravities and buoyancies.

According to an aspect of an embodiment of the invention t here is provided a magnetically responsive particle. The materials that make up the magnetically-responsive particle include a combination of MRM and base material. The base material may itself include one or more other materials, such as a material selected for low-density or high-density, a coating to inhibit interaction with surrounding fluid, a core such as a hollow or solid glass sphere, a matrix material to hold the particle together, a coating to control particle-to-particle interaction and others.

Other aspects and features of the present invention will become apparent, to those ordinarily skilled in the art, upon review of the following description of the specific embodiments of the invention.

### BRIEF DESCRIPTION OF THE DRAWINGS

For a better understanding of the present invention, and to show more clearly how it may be carried into effect, reference will now be made, by way of example, to the accompanying drawings, which illustrate aspects of embodiments of the present invention and in which:

FIG. 1 is a simplified schematic diagram of a system for evaluating a fluid system according to an embodiment of the invention;

FIG. 2 is a flow-chart illustrating a method of employing the system for evaluating a fluid system shown in FIG. 1 according to an aspect of the invention;

FIG. 3a is a simplified schematic diagram of a manufacturing system for manufacturing magnetically-responsive particles according to an embodiment of the invention;

FIG. 3b is a flow-chart illustrating a method of manufacturing magnetically-responsive particles employing the manufacturing system shown in FIG. 3a according to an aspect of the invention;

FIG. 4a is a first graphical illustration of the densities, of different magnetically-responsive particles as a function of a specified (ie. selected) mass ratio and base material density according to aspects of the invention;

FIG. 4b is a second graphical illustration of the densities, of different magnetically-responsive particles as a function of a specified (ie. selected) mass ratio and base material density according to aspects of the invention;

FIG. 5a is a cross-sectional view of a magnetic particle-collector according to an embodiment of the invention;

FIG. 5b is perspective view of a magnetic particle-collector according to another embodiment of the invention;

FIG. 6 is a perspective view of a magnetic particle-collector according to another embodiment of the invention;

FIGS. 7a and 7b are magnified face and side views respectively of a portion of a magnetic particle-collector grid according to another embodiment of the invention;

FIG. 8 is a transparent perspective view of a particle-collector array according to another embodiment of the invention;

FIGS. 9a is a perspective view of a magnetic particle-collector according to another embodiment of the invention;

FIGS. 9b and 9c are perspective views of a magnetic particle-collector shown in FIG. 9a with an optional protective box in closed and open positions;

FIG. 9d is a perspective view of a magnetic particle-collector according to another embodiment of the invention;

FIGS. 10a and 10b are perspective views of a magnetic particle-collector according to other embodiments of the invention;

FIG. 10c is a side view illustrating a way of using the magnetic particle-collector shown in FIGS. 10a and 10b;

FIG. 10d is a side view illustrating another way of using the magnetic particle-collector shown in FIG. 10a and 10b;

FIG. 11 is a side view illustrating another way of using the magnetic particle-collectors shown in FIG. 10;

FIG. 12a is a schematic illustrating a process for making magnetically-responsive particles in accordance with another embodiment of the invention;

FIG. 12b is a flow diagram illustrating steps in the process shown in FIG. 12a;

FIG. 13a is a schematic illustrating a process for making magnetically-responsive particles in accordance with another embodiment of the invention;

FIG. 13b is a flow diagram illustrating steps in the process shown in FIG. 13a;

FIG. 14 is a schematic illustrating a process for making magnetically-responsive particles in accordance with another embodiment of the invention;

FIG. 15a is a schematic illustrating a process for making magnetically-responsive particles in accordance with another embodiment of the invention;

FIG. 15b is a flow diagram illustrating steps in the process shown in FIG. 15a;

FIGS. 16a-16d are views of magnetically-responsive particles in accordance with other embodiments of the invention;

FIG. 17 is a view of a magnetically-responsive particle in accordance with another embodiment of the invention;

FIG. 18 is a view of a magnetically-responsive particle in accordance with another embodiment of the invention;

FIGS. 19a, 19b and 19c are views of magnetically-responsive particles in accordance with other embodiments of the invention;

FIGS. 20a and 20b are photographic images showing an embodiment of the magnetically-responsive particle schematically described in FIG. 18;

FIG. 21 is a view of a magnetically-responsive particle in accordance with another embodiment of the invention;

FIGS. 22a and 22b are views illustrating the behavioural difference between magnetically-responsive particles in certain embodiments;

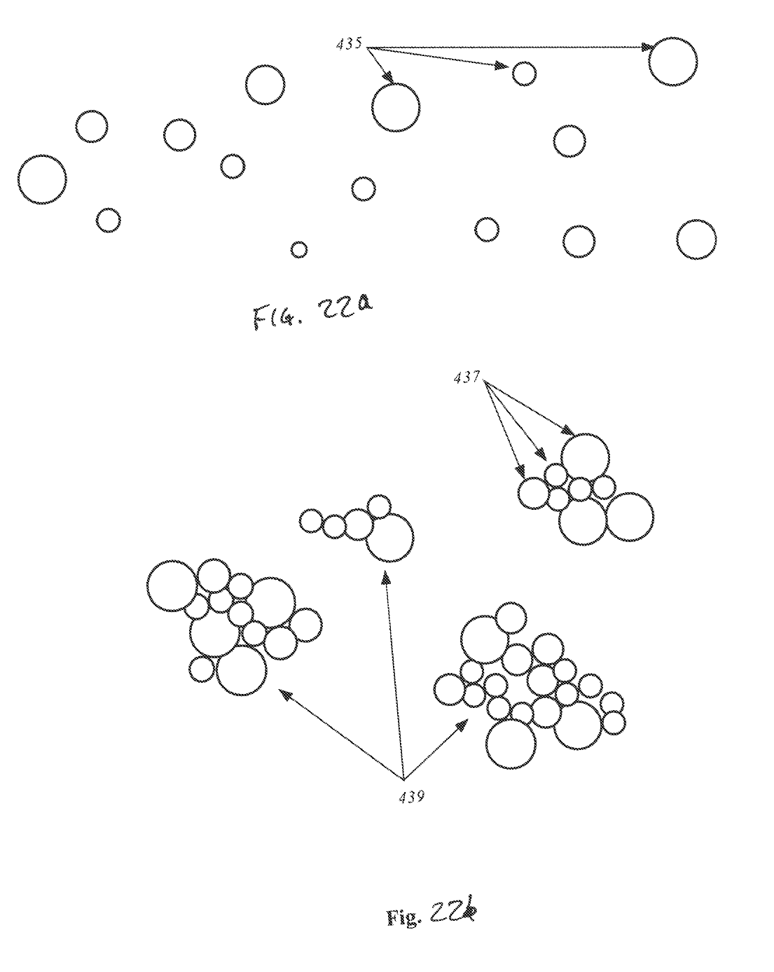

FIGS. 23a and 23b are photographic images of the magnetically-responsive particle shown in FIG. 16a;

FIG. 24 is a photographic image showing an example density distribution embodiment of the magnetically-responsive particles; and

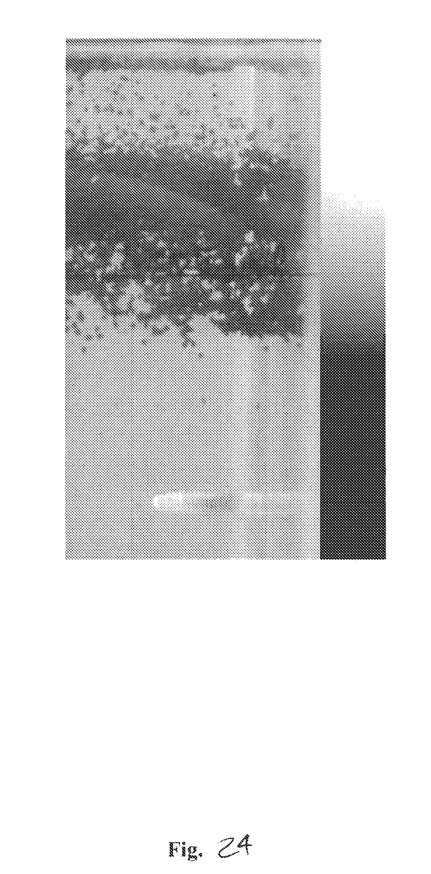

FIG. 25a, 25b and 26 show photographic images of a magnetic particle-collector according to an embodiment of the invention schematically illustrated in FIG. 6.

### DETAILED DESCRIPTION OF THE INVENTION

Conventional techniques and technologies, for collecting data to evaluate a fluid system are often prohibitively expensive in terms of equipment and survey (e.g. vessel) operating costs and the number of man-hours required to collect the data sought. In addition to the associated costs and time needed, many of the conventional techniques and technologies cannot be effectively employed to collect the types of data sought (e.g. tracing particulate dispersion through a coral system). In many scenarios the data that can be collected are incomplete and/or flawed. In many scenarios conventional surveys of the dispersion field do not allow all places in the field to be monitored at the same time, or over the same period. Moreover, specific variants of apparatus, systems and methods developed and/or adapted for environmental field applications are not always suitable for industrial applications, and vice versa.

In contrast, some embodiments of the invention provide a system for evaluating a fluid system, which includes one or more batches of magnetically-responsive particles and one or more magnetic particle-collectors. In some embodiments, the magnetically-responsive particles have a composition that includes elements that are attracted by magnetic fields, but do not themselves act as magnets and do not attract other magnetically-responsive particles. In some embodiments, the magnetically-responsive particles have a composition that includes elements that are attracted by magnetic fields and do themselves act as magnets to attract other magnetically-responsive particles. In some embodiments, the magnetic particle-collectors include and/or are constructed from magnetic elements, such as permanent magnets and electro-magnets shaped as bars, rods, horseshoes, rings or donuts, disks, rectangles, multi-fingered rings, and other custom shapes. Each batch of magnetically-responsive particles can be released into the fluid system (e.g. sea-water, a multi-phase sewage slurry, water produced from a drill rig or from erosional sediments and fluids) at a location upstream, adjacent to or in the vicinity of the expected dispersion field and upstream of the already-deployed, soon-to-be-deployed, or repeatedly deployed magnetic particle-collectors. The magnetically-responsive particles will follow fluid flows, experiencing effects, such as for example and without limitation, dispersion, diffusion, sinking and/or rising, mixing and advecting as they move with and through the fluid system. As magnetically-responsive particles reach the vicinity of the magnetic particle-collectors, the magnetically-responsive particles will be attracted into and held by the magnetic field and physical structure of the magnetic particle-collectors. Upon retrieval of the magnetic particle-collectors, the magnetically-responsive particles collected by each magnetic particle-collector are then quantified, and subsequently compared to the number of magnetically-responsive particles collected by other magnetic particle-collectors and the number of magnetically-responsive particles originally released, thereby providing a means for collecting information about the fluid system.

As a very specific example, one application of an embodiment of the invention is determining the so-called “ecological connectivity” of a marine system. Ecological connectivity is sometimes defined as the rate at which marine plants and animals and their propagules move among locations and it is necessary to determine the probability that propagules from one or more location(s) will arrive at one or more same or different location(s) over some period. The example of the magnetically-responsive particle and magnetic particle-collector system described hereinafter can be arranged to collect the information necessary to estimate these probabilities. One specific embodiment of this application related to this concept may include a mechanism to release magnetically-responsive particles slowly over time to obtain a better statistical average over the variety of flow conditions found in a particular fluid system in a certain geographical area or industrial setting.

In accordance with some embodiments for specific applications, the effective distance over which the magnetic attraction (from the magnetic particle-collectors) occurs is in the range of a few centimeters. By contrast the fluid paths that the magnetically-responsive particles will likely travel ranges from several meters to hundreds (possibly thousands) of kilometers. Accordingly, the magnetic attraction and placement of the magnetic particle-collectors is unlikely to affect the fluid path followed by the magnetically-responsive particles, except for the final few centimeters, which is relatively insignificant compared to the much larger distances of the fluid paths in the fluid system being analyzed.

In some embodiments, the system provided can be employed to make dispersion measurements. For example, employing a system according to an embodiment of the invention may provide the possibility of direct observation of sub-grid scale dispersion, which is a poorly known quantity that is advantageous to accurately parameterising, developing and improving the performance of numerical models. As noted above, typical current measurements (flow sensor arrays, conventional drifters etc.) provide information at scales inappropriate to providing this information.

In some embodiments a particular batch of magnetically-responsive particles are manufactured, according to a manufacturing process provided by an aspect of the invention, so that the particular batch has a respective specific gravity distribution, a respective buoyancy distribution, and a respective size distribution. That is, in some embodiments, each batch of particles is manufactured such that each particle has a respective specific gravity value, a buoyancy value and a size value that is or is very close to a corresponding specified (ie. selected)specified (ie. selected) (ie. selected) specific gravity value, a specified (ie. selected)specified (ie. selected) (ie. selected) buoyancy value and a specified (ie. selected)specified (ie. selected) (ie. selected) size value. However, due to manufacturing inaccuracies not all magnetically-responsive particles can be perfect, so distributions around the aforementioned specified (ie. selected) values are specified (ie. selected) instead. Specifications can be, but are not limited to, the rise or sinking rates of the magnetically-responsive particles and their distribution as a function of the fluid density and/or its distribution. In more specific embodiments, each batch of magnetically-responsive particles also has a specified (ie. selected) color or dye or fluorescent dye-component that is visible under light of a given wavelength such as full-spectrum visible to the eye or visible to the eye when exposed by ultra-violet wavelengths to further enable dispersion tracing. In other more specific embodiments, each batch of magnetically-responsive particles has a composition that includes a base material that is biodegradable, and specified (ie. selected) to breakdown after a known period of time. In various embodiments, the buoyancy of the magnetically-responsive particles is chosen so that the magnetically-responsive particles are, on average, positively buoyant, neutrally buoyant, or negatively buoyant. In various embodiments, the size of the magnetically-responsive particles is specified (ie. selected) to be in the range of 5 microns to 20 mm. In some embodiments, the magnetically-responsive particles include an inert and/or non-toxic base material. In some embodiments, the magnetically-responsive particles include an additive to make them resistant to bio-fouling.

In some embodiments, the magnetic particle-collectors include a hollow body and/or open-ended net for concentrating (e.g. mesh-filter funnel) magnetically-responsive particles in the flow and around or near the magnetic elements on and/or within the magnetic particle-collector and to attract and trap the magnetically-responsive particles. In some more specific embodiments, the magnetic particle-collectors can be free-floating, towed (e.g. side-towed) anchored and/or moored in a particular location in the fluid. In some specific embodiments, the magnetic particle-collectors are tubular and open-ended. In some embodiments the magnetic particle-collectors are deployed as coring devices used to penetrate sediments or as harrow devices used to scour sediments. In more specific embodiments the open end of the tubular magnetic particle-collector includes a flow control device (e.g. a passive or active valve, such as a ball valve) that is provided to control the maximum velocity of fluid through the magnetic particle-collector. Additionally and/or alternatively, in some embodiments, a magnetic particle-collector includes a cone-shaped net, open at both ends and arranged to concentrate magnetically-responsive particles that enter the net, causing the magnetically-responsive particles to flow closely past, be attracted to and retained by the magnetic element(s). A magnetic particle-collector may include any suitable number of magnets in any arrangement in or on the magnetic particle-collector, including flow-through or flow-by magnetic element configurations, as well as single or multiple magnets in a linear (string) or matrix (netting) array, with the magnets connected together or to the magnetic particle-collector using any suitable structure, fabric or adhesive. The magnetic particle-collector are designed to be set out (moored, suspended, free-standing, free-drifting, towed or deployed for sediment coring or sediment harrowing) in a fluid environment for a period, after which the number of magnetically-responsive particles that have been collected is quantified. Additionally and/or alternatively, the number of magnetically-responsive particles that accumulate in or on the magnetic particle-collectors can be quantified periodically at intervals within a period for multiple periods. A flow-meter may be associated with a magnetic particle-collector to monitor variations in fluid flow.

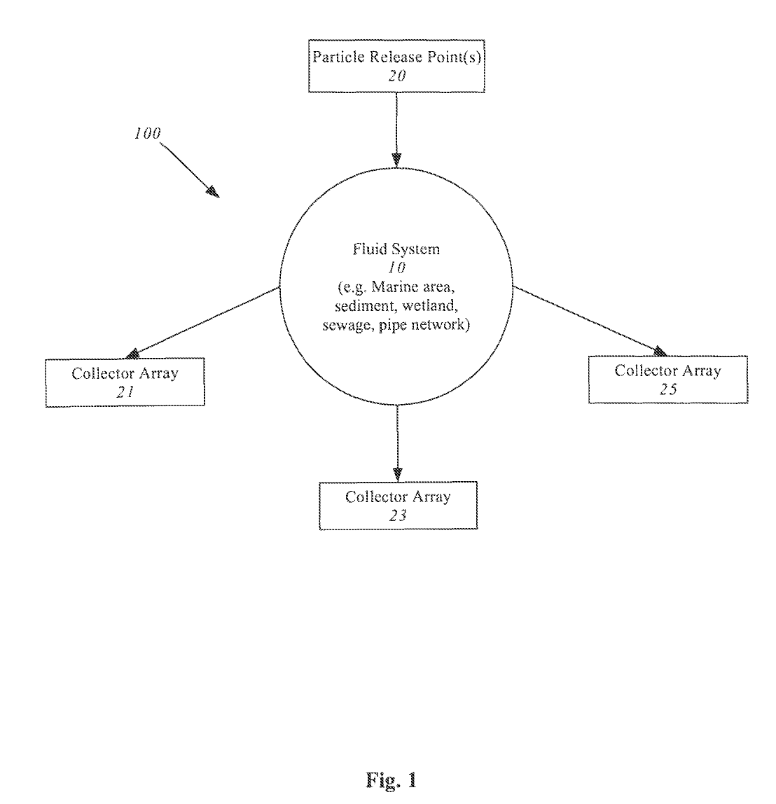

Referring to FIG. 1, shown is a simplified schematic of an example system 100 for evaluating a fluid system 10 according to an embodiment of the invention. The system 100 includes at least one magnetically-responsive particle release point 20, where magnetically-responsive particles not shown) are released into the fluid system 10, and at least one magnetic particle-collector for collecting magnetically-responsive particles from the fluid system 10. More specifically, the system 100 includes three magnetic particle-collector arrays 21, 23, 25 that each includes at least one magnetic particle-collector (not individually shown). Although only the three magnetic particle-collector arrays 21, 23, 25 are shown, it should be emphasized that the system 100 is provided only as an example, and any number of magnetic particle-collector arrays or individual magnetic particle-collectors and any number of magnetically-responsive particles released from the same location at different times or at the same time, and/or different locations at the same time or at different locations at different times could be included in alternative systems without departing from the scope of the claims following this section.

The fluid system 10 is the environment to which the system 100 is applied and it may be any number of natural, manmade and industrial environments and/or any combination thereof. The fluid system 10 could be examined to validate a numerical flow model and/or determine a number of characteristics of the fluid system 10 depending on the specific information sought, including without limitation, fluid flow paths, dispersion, diffusion and advection of particulates, biological propagules, toxins, contaminants or sediments and the like, the extent of environmental impact of the like etc. For example, the fluid system 10 can be examined using the system 100 to trace the dispersion of suspended biological propagules (e.g. eggs, larvae, etc.), particulates, pollutants, toxins, natural and manmade dyes and sediments through the fluid system 10. Examples of fluid systems that may be analyzed using embodiments and according to aspects of the invention, include, without limitation, constrained fluid flows, rivers, lakes, wetlands, estuaries, harbors, coastal and open ocean systems, industrial pipelines (and piping), wells, drill-rigs, beaches, erosional studies, bedform transport studies, aquaculture studies, deep-sea hydrothermal vents and cold-seep eco-systems, sewage systems, storm drainage systems, irrigation systems, underground water systems and reservoirs and/or any combination thereof.

As noted above, each batch of magnetically-responsive particles is manufactured such that each magnetically-responsive particle has a respective specific gravity value, a buoyancy value, a rise or sinking rate value and a size value that is or is very close to a corresponding specified (ie. selected) specific gravity value, a specified (ie. selected) buoyancy value, a specified (ie. selected) rise or sinking rate value and a specified (ie. selected) size value. Each of these values can be manipulated independently to create batches of magnetically-responsive particles with desired characteristics. For example, in some specific embodiments the specified (ie. selected) buoyancy value corresponds to a buoyancy value that is relative to the fluid they are released into. Subsequently, as will be described in more detail below, the magnetically-responsive particles either float, are suspended, or sink in the fluid depending on the specified (ie. selected) buoyancy value desired.

In a more specific example, if the magnetically-responsive particles are used to study the dispersion pattern of biological propagules such as coral larvae, the specified (ie. selected) buoyancy and specified (ie. selected) size of the magnetically-responsive particles is chosen such that the average buoyancy and size of the magnetically-responsive particles matches that of the early-stage coral larvae (which are approximately neutral to slightly-positively buoyant in sea-water), so that the magnetically-responsive particles follow the same fluid paths as the coral larvae. In another example, the magnetically-responsive particles are manufactured to be negatively buoyant so that the magnetically-responsive particles sink in a fluid system and can then be transported via turbulent motions of bottom currents, if at all. In another example the magnetically-responsive particles are manufactured to be negatively buoyant so that the magnetically-responsive particles behave as sediments of a specified (ie. selected) character (size, density, buoyancy, sinking rate) in a fluidized sediment environment and can then be bedform transported. A method, according to an aspect of the invention, for manufacturing the magnetically-responsive particles for various specific applications is described below.

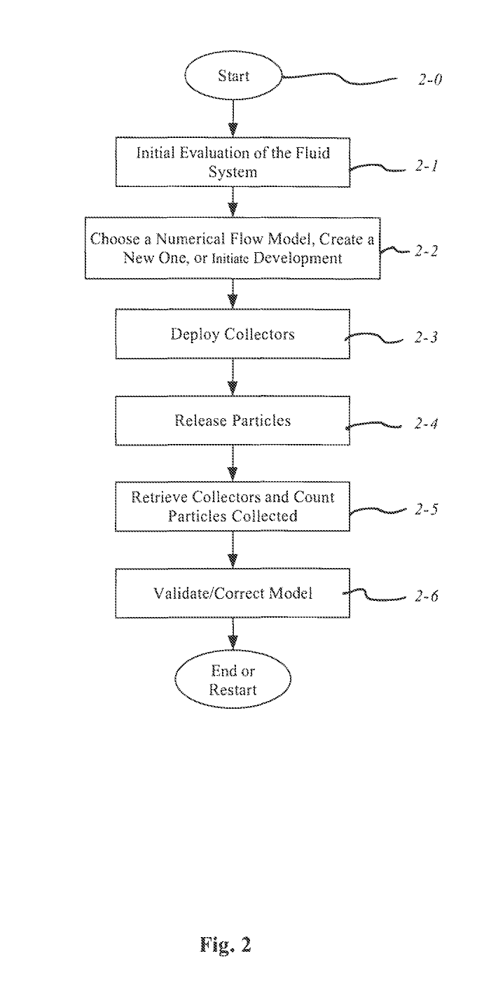

Referring now to FIG. 2, and with continued reference to FIG. 1, shown is a flow-chart illustrating a method of employing the system 100 for evaluating the fluid system 10 according to an aspect of the invention. Starting at step 2-1, the fluid system 10 undergoes an initial evaluation in which information, which does not necessarily depend on the fluid flow paths through the fluid system 10, is compiled and with assumptions and/or theories about the fluid system 10. Such information may include, without limitation, structural diagrams, structural or geological information, environmental or weather information, fluid current and density information and the presence of pollutants, toxins, particulates and the like at various locations in the fluid system. At step 2-2, a numerical flow model is chosen, created or developed and further developed, validated and/or corrected at later steps.

At step 2-3 a number of magnetic particle-collector arrays (e.g. magnetic particle-collector arrays 21, 23, 25—see FIG. 1) are deployed throughout the fluid system 10, based on the known and assumed characteristics of the fluid system 10, to collect magnetically responsive particles. At step 2-4, one or more batches of magnetically-responsive particles are released at one or more locations with the fluid system 10. The magnetically-responsive particles are then moved by and through fluid flow paths that exist within the fluid system 10. Eventually, some of the magnetically-responsive particles are expected to reach at least one of the magnetic particle-collector arrays where magnetic particle-collectors trap them. The number of magnetically-responsive particles trapped in each magnetic particle-collector is quantified after the magnetic particle-collectors are retrieved, at step 2-5. If none of the magnetically-responsive particles reach any of the magnetic particle-collectors, then the evaluation must be started again at step 2-1, taking this information into account in the design of the new evaluation.

It should be noted that the amount of time between releasing the magnetically-responsive particles at step 2-4 and retrieving the magnetic particle-collectors at step 2-5 (to quantify the collected magnetically-responsive particles) can be deliberately varied to monitor the effect of time on the flow of magnetically-responsive particles through the system. Additionally and/or alternatively, the number of magnetically-responsive particles collected can be quantified periodically at intervals over a sample period to collect more information about the fluid system 10, than if the number of magnetically-responsive particles collected was quantified only once.

After the raw data relating to the magnetically-responsive particles collected by different magnetic particle-collectors is obtained at step 2-5, at step 2-6 the numerical flow model chosen or created or under development at step 2-2 can be validated and/or corrected or further developed by processing the raw data. According to some aspects of the invention, the data processing employs statistical methods of relating the raw data to the numerical flow model chosen, created or under development. Additionally and/or alternatively, it is entirely possible, that a new numerical flow model can be created using the raw data, previously known information, and assumptions that better support the empirical information represented by the raw data.

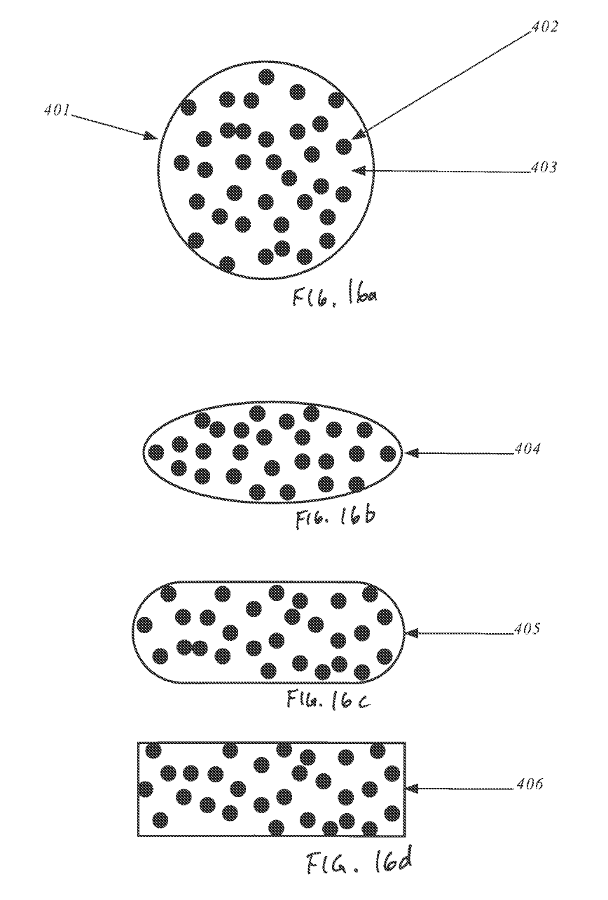

In accordance with some embodiments and aspects of the invention, a specific process for manufacturing magnetically-responsive particles includes extrusion and pelletising of a composite melt through die-plates of varying mesh-size at varying pressures and subsequent cooling such that the magnetically-responsive particles produced are of the desired size (e.g. ranging from approximately 5 microns to approximately 20 mm in diameter). FIG. 16a illustrates such a particle 401 containing a magnetically-responsive material, MRM, 402 in a base material 403 in specified (ie. selected) proportions so as to result in a specified (ie. selected) target density. Additionally and/or alternatively, the composite melt is extruded to produce various shapes such as, but not limited to, rods, cylinders or fibers of a desired diameter and cross-section that can be size-reduced (e.g. by cutting and/or shaving and/or grinding and/or any other suitable size-reduction means) to produce the magnetically-responsive particles of the desired dimensions. In accordance with some aspects of the invention, shape variations between batches of magnetically-responsive particles can be achieved through the extrusion process (melt temperature, extrusion pressure etc.), mesh-size and shape of extrusion plates, free-fall and cooling processes. FIG. 16 illustrates some of a number of possible magnetically-responsive particle shapes 404, 405, 406 that can be produced by the above variations of the manufacturing process. In accordance with some aspects of the invention, color variations can be by the addition of fluorescent-dye additives (e.g. rhodamine, fluorescine, etc.) and color-dye additives. Color may be added to some or all of the magnetically-responsive particles. Some of the magnetically-responsive particles may have different colors than others.

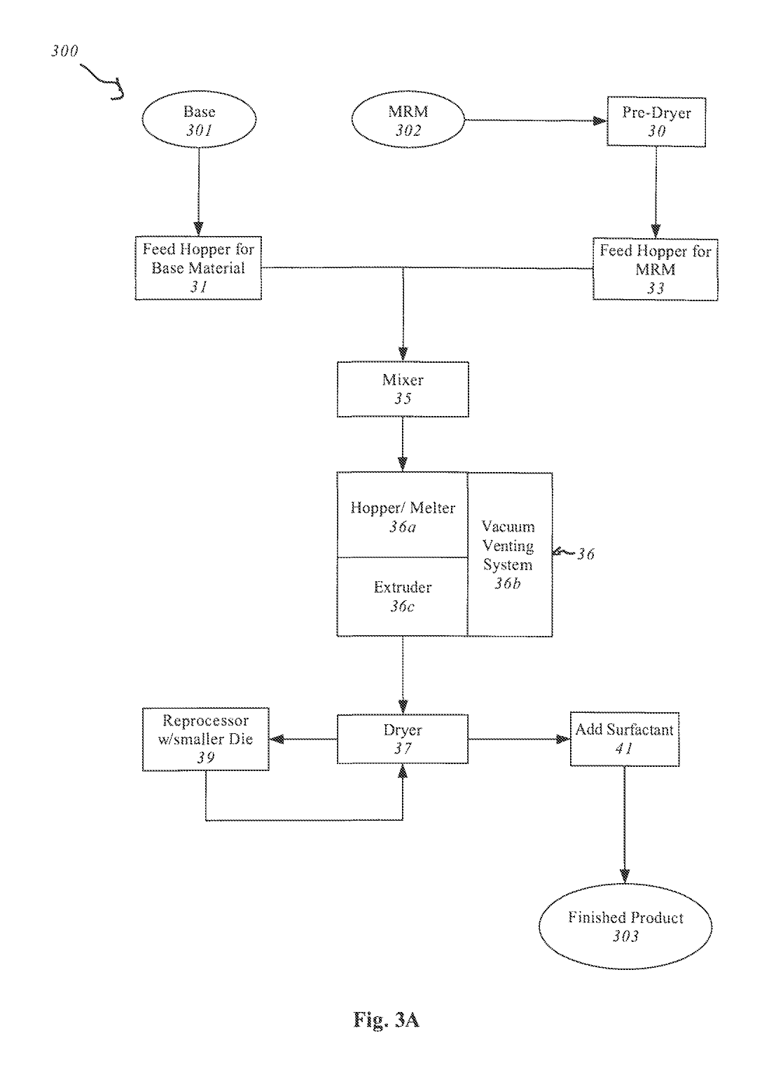

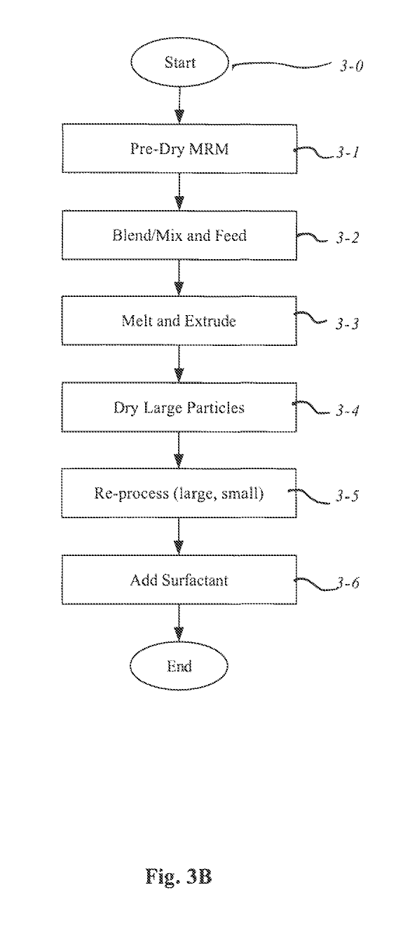

Turning to FIGS. 3a and 3b, shown is a simplified schematic diagram of a manufacturing system 300 and a corresponding flow-chart illustrating a method of manufacturing magnetically-responsive particles, employing the manufacturing system 300, according to an embodiment and aspects of the invention. The manufacturing system 300 and manufacturing process are described in tandem for the sake of brevity.

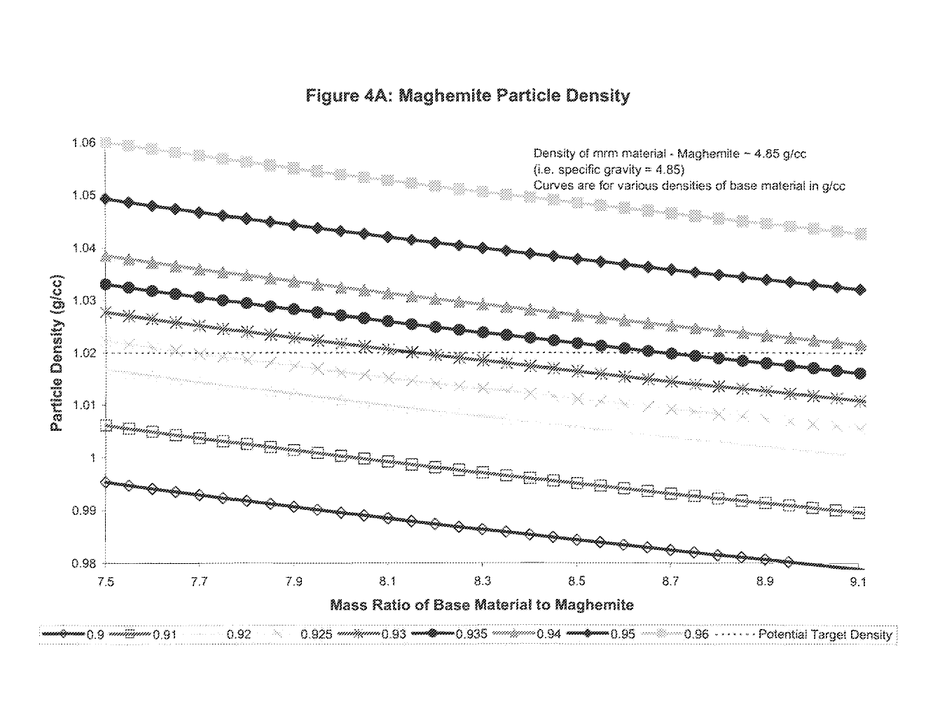

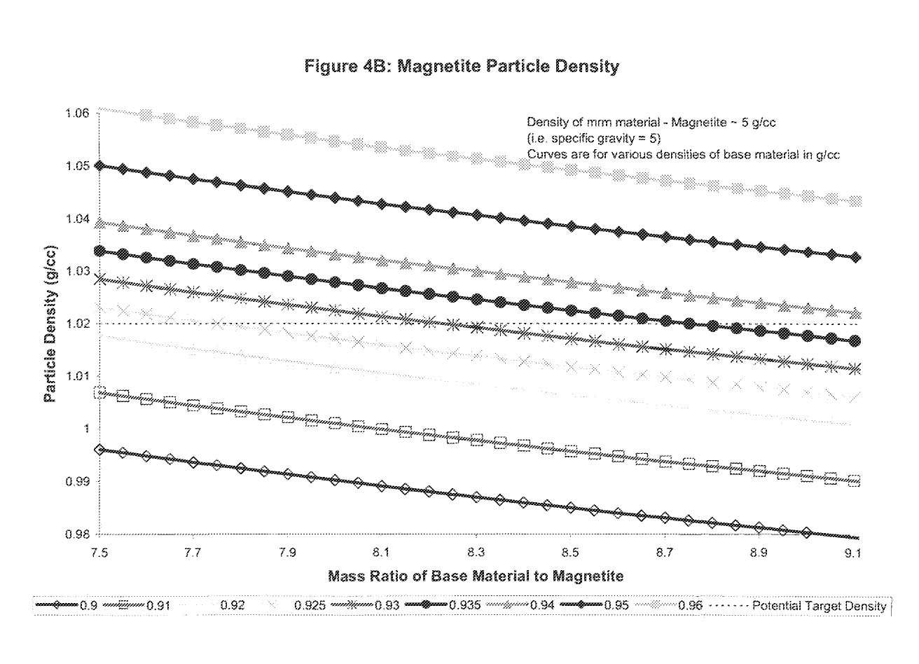

In some embodiments, the magnetically-responsive particles are manufactured from a lower density base material 301 and magnetically-responsive material (MRM) 302. Specific examples of the composition of the magnetically-responsive particles are described in greater detail below with respect to FIGS. 4a and 4b. However, briefly, at this point, it is worth noting that MRMs typically have a higher density than the fluids (e.g. water) in the system to be studied. Accordingly, such materials are expected to sink in the fluid unless they are manufactured as buoyant pieces. The manufacturing of specifically shaped and specifically buoyant pieces on a large scale is usually prohibitively expensive and the risk of flaws in the shapes and deviations from the specified (ie. selected) buoyancy is often too great. Consequently, it is preferable to employ MRMs in a particulate or powder form, in which individual particulates (in the range of 1-10 micron) can be assumed to be uniformly dense and solid. However, the individual pieces of such particulates and powders are negatively buoyant in many fluids (e.g. fresh water, distilled water, sea-water, mixed-phase sewage slurries, alcohols, petroleum products etc.) and will sink. In accordance with some aspects of the invention an MRM particulate or powder is combined with a base material that is less dense than the fluid in the fluid system to create magnetically-responsive particles of desired buoyancy. By combining the base material 301 and the MRM particulate or powder 302 in appropriate proportions, magnetically-responsive particles can be manufactured. Again, examples of various compositions are described in greater detail below with reference to FIGS. 4a and 4b.

Referring back to FIG. 3a, the manufacturing system 300 includes a pre-dryer 30, a first feed hopper 31 for the base material 301, a second feed hopper 33 for the MRM 302, a mixer 35, an extrusion system 36, a dryer 37, a re-processor 39 and a surfactant wash 41.

The pre-dryer 30 is employed to dry the MRM before further processing. Some MRMs include powdered magnetite (Fe₃O₄) and maghemite (γFe₂O₃), which have respective water contents in the range of 1-2%, which is preferably reduced before further processing. Accordingly, with additional reference to FIG. 3b, starting at step 3-1, the powdered MRM is dried in the pre-drier 30 (FIG. 3a) to reduce the water content. The dried output of the pre-dryer 30 is fed to the second feed hopper 33. The base material may or may not have to be pre-dried as well. For example, a base material like polyethylene powder may have water content in the range of 0.2% which does not have to be reduced before further processing. In the embodiment shown in FIG. 3a, a pre-drier for the base material is not shown. The reason it is preferable to reduce the water content of the base material (if it is too high) and of the MRM (if it is too high) is that as the water evaporates in the extrusion system 36 it produces tiny gas bubbles in the melted composition of base material and MRM, which adversely effects control over the buoyancy of the magnetically-responsive particles. This aspect will be described in greater detail below with reference to FIGS. 4a and 4b.

The first and second feed hoppers 31, 33 (FIG. 3a) are arranged to provide the base material and the MRM, respectively, to the mixer 35. In an automated process, the mixer 35 can be operated to control the ratio at which the base material and MRM are blended and delivered to the extrusion system 36. Employing the mixer 35 in this manner is preferable as it helps to reduce differential sefting that may occur if the base material and MRM are simply mixed in bulk and fed to the extrusion system 36. With additional reference to FIG. 3b, at step 3-2 the base material and MRM are blended/mixed in the mixer 35 (FIG. 3a) and fed to the extrusion system 36. The terms blend and mix are used interchangeably in this disclosure to describe the mixing of the base material and the MRM.

The extrusion system 36 includes a hopper/melter 36a, a vacuum venting system 36b and an extruder 36c. The hopper/melter 36a is employed to melt polymer-based base material so that the base material envelops the MRM particulates in the composition to produce a melt that is substantially free of gas and moisture bubbles. The vacuum venting system 36b, pulls away gases and water vapor released in the melting, mixing and extrusion process to ensure that the released gases and water vapor are not reincorporated into the melt. The vacuum venting system 36b also aids in alleviating back pressure from the extruder 36c. The extruder 36c may be, for example and without limitation, a 26 mm twin-screw extruder fitted with an underwater pelletiser (not shown) having a 2.8 mm die. As illustrated in FIG. 3b the melt is processed and extruded to produce relatively large magnetically-responsive particles at step 3-3, which are then dried in the drier 37 at step 3-4.

At step 3-5, (FIG. 3b), after being dried the relatively large magnetically-responsive particles are re-processed by the re-processor 39 (FIG. 3a), having a substantially smaller die (e.g. a 300-500 micron die), to produce smaller magnetically-responsive particles. In some embodiments, the re-processor 39 shares the same extruder as the extrusion system 36. Additionally and/or alternatively, a separate extruder may be employed. The smaller magnetically-responsive particles are then sent through the surfactant wash 41 at step 3-6. The addition of a surfactant to the magnetically-responsive final product reduces clumping of magnetically-responsive particles when they are later released into a fluid system. Additionally and/or alternatively, the smaller magnetically-responsive particles can be created in a single step, without first creating the larger magnetically-responsive particles. However, depending on the specific equipment employed, managing the percentage of defective magnetically-responsive particles in the final product is sometimes difficult. Moreover, the back-pressure created when trying to produce very small magnetically-responsive particles in a single step is sometimes too much for some equipment to handle and so a multi-step process is better in such cases. Nonetheless, producing very small magnetically-responsive particles in a single step is within the scope of some embodiments of the invention.

Referring to FIG. 16 illustrates a particle 401 containing magnetically-responsive material 402 in a base material 403 in specified (ie. selected) proportions to produce a magnetically-responsive particle of the specified (ie. selected) target density. FIG. 16 also illustrates for example and without limitation different magnetically-responsive particle shapes 404, 405, 406 that can be produced by the above variations of the manufacturing process.

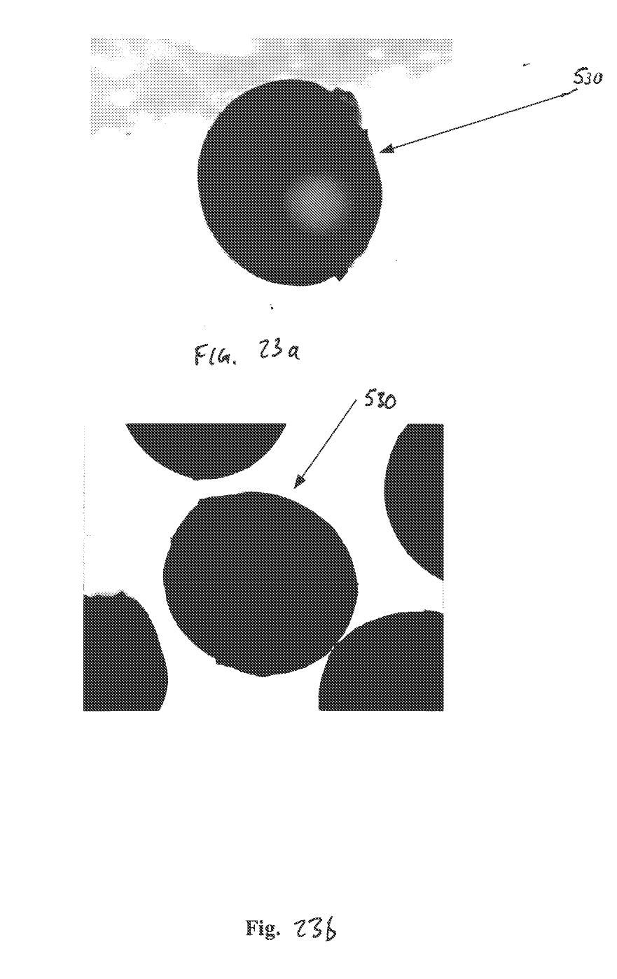

Referring to FIGS. 23a and 23b show an actual magnetically-responsive particle 530 photographed under a microscope of a spherical embodiment of a magnetically-responsive particle manufactured using the above mentioned manufacturing process.

As noted above, in accordance with some embodiments of the invention, the composite magnetically-responsive material that magnetically-responsive particles are manufactured from includes a low-density base material and a higher-density MRM, such as a magnetically-responsive particulate or powder. By combining the base material and the MRM in specified (ie. selected)selected ratios (based on mass and density) magnetically-responsive particles can be manufactured that have a specified (ie. selected) buoyancy distribution around a particular buoyancy value. Variations of the above components' mixture ratios and melt-flow indices of the base material, including gas-injection and/or heating or cooling to the material during mixing and/or extrusion and the MRMs are used to produce a composition that has a target density between 0.1-4 kg/m³.

Examples of the base materials that may be employed in the composition of the magnetically-responsive particles include, without limitation, various densities of waxes (e.g. paraffin), plastics, polymers and plastic polymers (e.g. thermoplastic polyamides, polyacrylamides, polystyrenes, polyethylenes polyurethanes etc.) low-density natural materials and mixtures (e.g. cellulose and celluloses aggregates) and low density manmade material such a hollow spheres of various sizes and material such a glass or ceramic. In some embodiments, the base material includes a self-solidifying resin and/or epoxy. Examples of the MRMs that may be employed include, without limitation, various magnetically attracted/responsive metallic materials and alloys (including ferrous and ferric iron and other iron-base alloys), magnetite and magnetite alloys, maghemite and maghemite alloys and other ferromagnetic and paramagnetic materials.

In specific embodiments, the base material includes a plastic, polymer, plastic or wax material and the MRM is in a powdered form. In such embodiments, the combination of the base material and the MRM can be assumed to have the combined volume of the base material and the MRM. That is, generally, there is no loss of volume when combining the base material with the MRM, when the base material is one of a plastic, polymer and wax with insignificant water content. This assumption is does not always hold, as would be the case when natural fibers (that sometimes have a higher moisture content and absorb melted waxes, plastics and the like) are included in the composition. It is also assumed that there is no loss of mass. This assumption generally holds if the moisture content of the various materials in the composition have been thoroughly dried and have almost zero (e.g. less that 0.5%) water content.

When the above assumptions do hold, the mass ratio (rₘ) of the base material to the MRM can be determined according to equation (1), given as follows:

$${r}_{m} = \frac{\frac{{ρ}_{mrm} - {ρ}_{t}}{{ρ}_{mrm} {ρ}_{t}}}{\frac{{ρ}_{t} - {ρ}_{b}}{{ρ}_{t} {ρ}_{b}}} \qquad ( 1 )$$

In equation (1), ρₜ is the target density for the magnetically-responsive particles, ρ_{b} is the density of the base material (e.g. paraffin wax), and ρₘᵣₘ is density of the magnetically-responsive material (MRM).

Referring to FIGS. 4a and 4b, shown are respective first and second graphical illustrations of the densities, of different magnetically-responsive particles, as a function of specified (ie. selected) mass ratios and base material density according to aspects of the invention. Specifically, FIG. 4a shows a graphical illustration of a range of particle densities (ρₜ) for magnetically-responsive particles having a various compositions, including waxes, at different densities and maghemite (γFe₂O₃) combined at various mass ratios rₘ as determined from equation (1). Similarly, FIG. 4b shows a graphical illustration of a range of particle densities (ρₜ) for magnetically-responsive particles having a various compositions, including waxes, at different densities and magnetite (Fe₃O₄) combined at various mass ratios rₘ as determined from equation (1).

In general a process for making the magnetically-responsive particles may include providing an MRM having an MRM density, providing a base material having a base material density, to combine with the MRM to form a combined material, wherein the ratios of the MRM and the base material are adjusted to provide the resulting magnetically-responsive particles with a specified (ie. selected) density, relative to the density of the fluid in which the magnetically-responsive particles will be placed. Combining the base material and the MRM may be achieved in any suitable way. For example, they may be mixed in a mixer, polymerized and formed into magnetically-responsive particles. It will be understood that, in such an embodiment, any portions of the resulting magnetically-responsive particles aside from the MRM would be defined as being base material.

Alternatively, the MRM may be provided as part of a coating that is applied to core particles to form magnetically-responsive particles. In such an embodiment, any portions of the magnetically-responsive particles aside from the MRM would be defined as being base material.

It will be appreciated that the base material may be made up of a plurality of constituent materials, having a plurality of densities. Some of the constituent materials that make up the base material may be part of the coating and some may be in the core particles. Additionally, some of the constituent materials may have a density that is higher than the target density and some may have a density that is lower. The ratios of the materials that make up the magnetically-responsive particles are selected so that the magnetically-responsive material has a specified (ie. selected) density, buoyancy, and a specified (ie. selected) rise rate or fall rate in the fluid system to be studied.

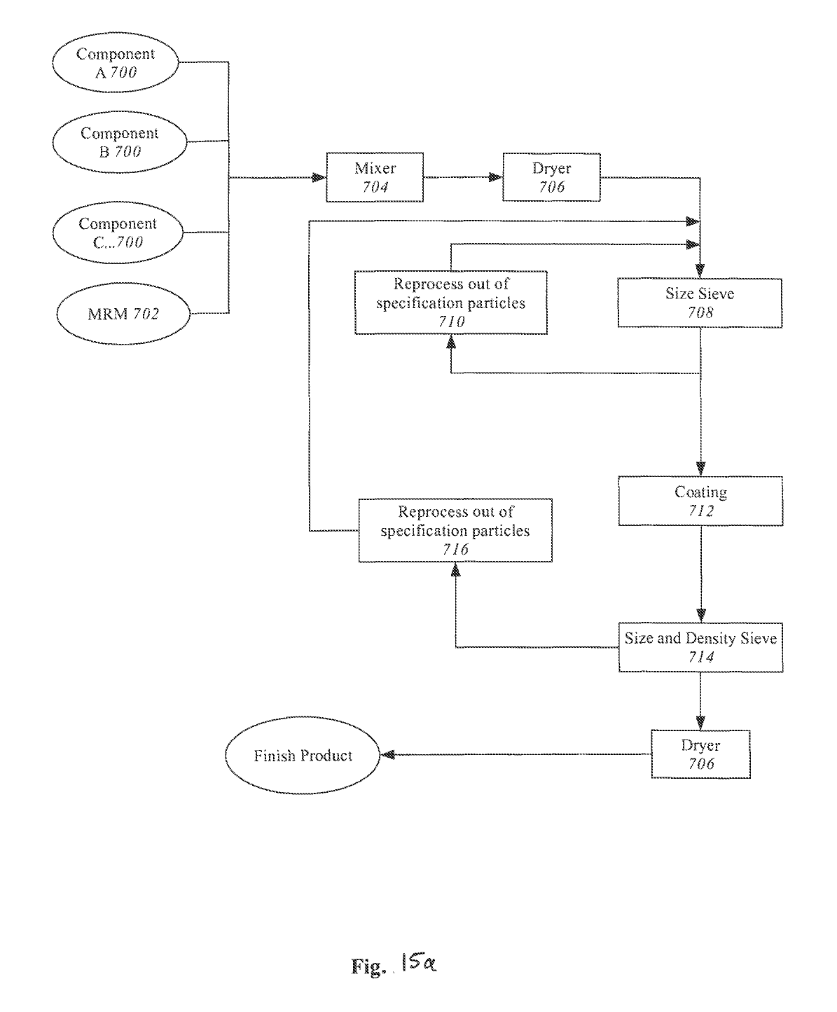

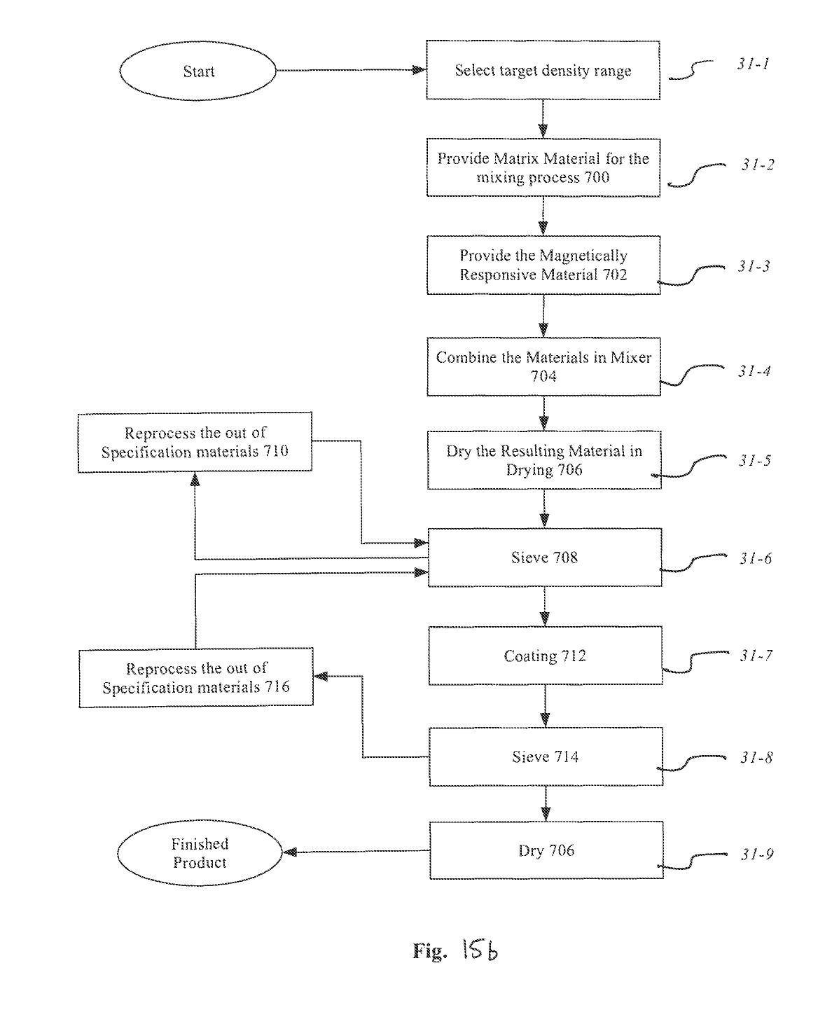

Reference is made to FIGS. 15a and 15b. In accordance with an embodiment of the invention, an exemplary process for manufacturing magnetically-responsive particles made up of different components includes steps 31-1 to 31-9.

Step 31-1 (FIG. 15b) is selecting a target final density range for the magnetically-responsive particles.

Step 31-2 (FIG. 15b) is providing materials that make up a matrix material 700 (FIG. 15a) in the required ratios to achieve a specified (ie. selected) size and density range that may be lower or higher than the final target density range.

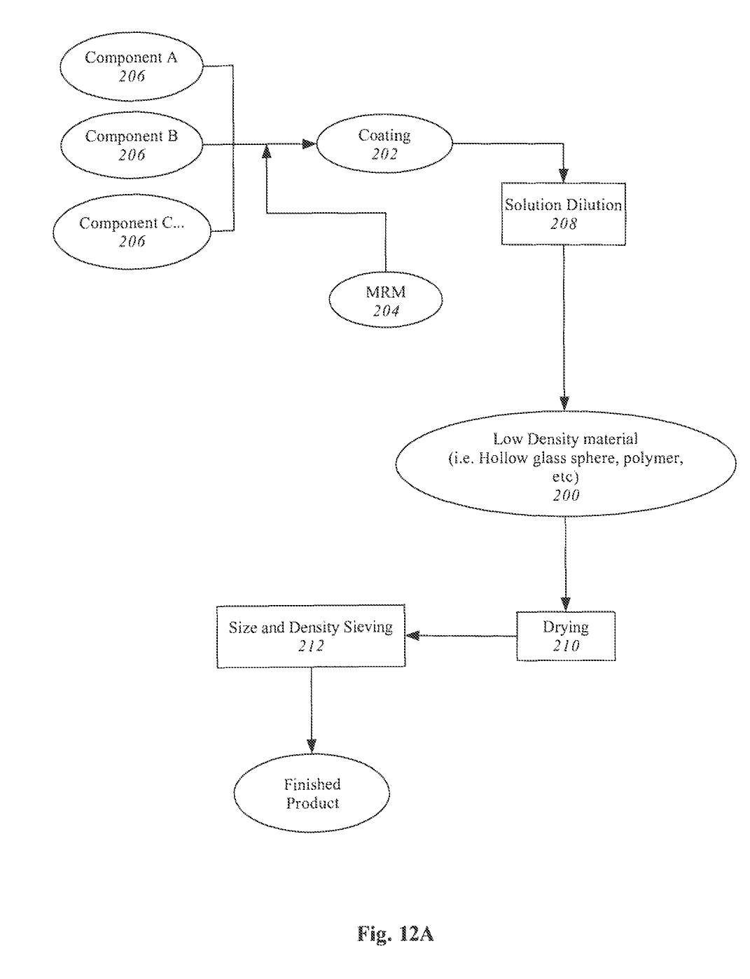

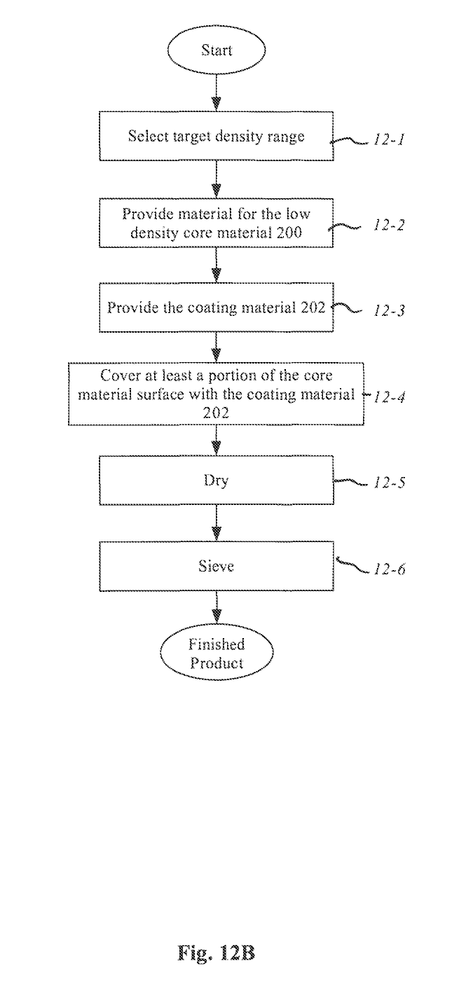

Step 31-3 (FIG. 15b) is providing a second material 702 (FIG. 12a) which is an MRM which has a density range that is at or higher than the final target density range.

Step 314 (FIG. 15b) is mixing the MRM 702 (FIG. 12a) and the matrix material 700 (FIG. 15a) at a specified (ie. selected) ratio required to achieve a specified (ie. selected) size and density range specified (ie. selected) for the final magnetically-responsive particles. Mixing may be achieved by using any suitable mixer, such as the mixer 704 in FIG. 15a.

Step 31-5 (FIG. 15b) is a drying process of the magnetically-responsive particles accomplished in the dryer 706 in FIG. 15a.

Step 31-6 (FIG. 15b) is sieving of the dried material such that particles of a specified (ie. selected) size or size range are retained for further processing described below. The large particles not retained by sieving may be reprocessed through size reduction means (e.g. grinding or crushing) and reintroduced at step 31-6.

Step 31-7 (FIG. 15b) is a coating process 712 whereby a coating material such as a polymer or an epoxy is used to modify the density characteristics of the magnetically-responsive particles.

Step 31-8 (FIG. 15b) is the size and density screening of the material from 31-7 above such that magnetically-responsive particles of a specified (ie. selected) size or size range are retained by sieving 714, and of those, subject to density selection based on buoyancy characteristics in a fluid of known density 714. Magnetically-responsive particles outside of the specified (ie. selected) ranges can be reprocessed 716 by re-introducing at step 31-6.

Step 31-9 (FIG. 15b) is a final drying 718 of the magnetically-responsive particles.

The first material may include, for example, hollow glass or ceramic spheres or shapes, hollow or solid polymer spheres or shapes, polystyrene or sawdust. The MRM may be non-dried, but is preferably dried. The mix of first material and MRM may be combined with a matrix material such as a multi-part-epoxy, cellulose, paper, or ceramic. The matrix material may polymerize when a reagent is added. By controlling the ratios of the materials and the mixing speeds the rate of polymerization can be controlled and the materials can produce magnetically-responsive particles of a specified (ie. selected) size with a relatively uniform specified (ie. selected) density.

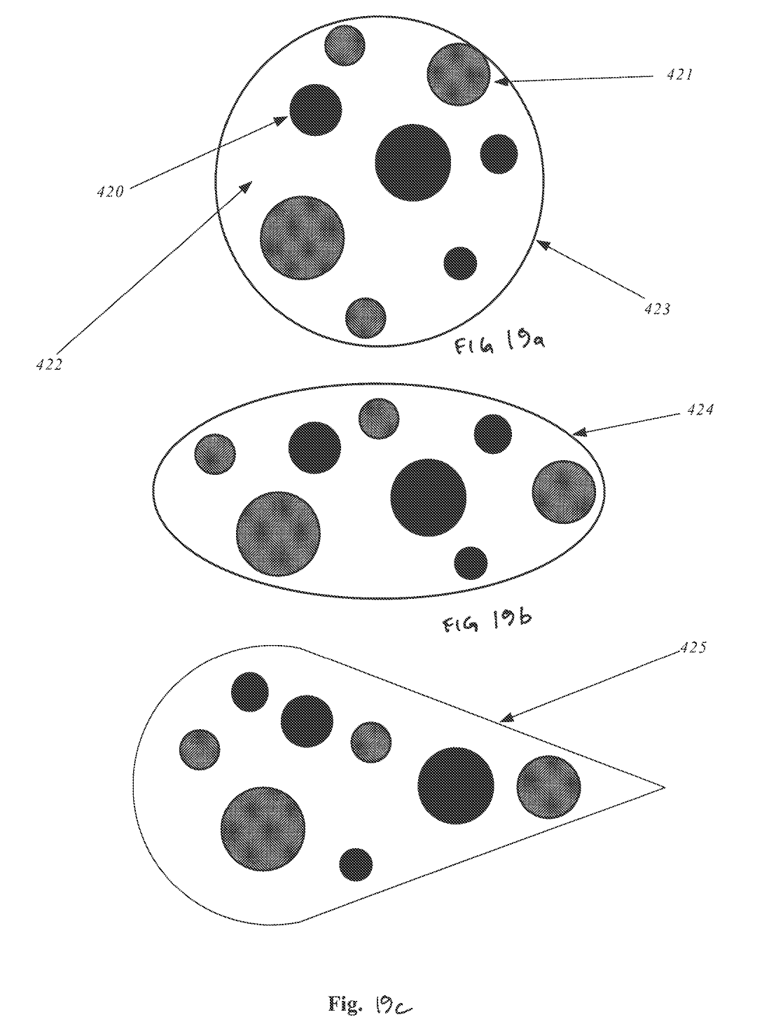

Referring to FIG. 19a, illustrating the magnetically-responsive particles produced by the process described by FIGS. 15a and 15b. The magnetically-responsive material 420 is mixed with a low density material 421 and encased in a matrix material 422 that is caused to polymerize so as to form magnetically-responsive particles 423 of specified (ie. selected) density. In alternative embodiments, the magnetically-responsive particles may include without limitation other shapes such as ellipsoidal 424 (FIG. 19b) or teardrop 425 (FIG. 19c), such that the various shapes can be used to identify magnetically-responsive particles from different manufacturing batches.

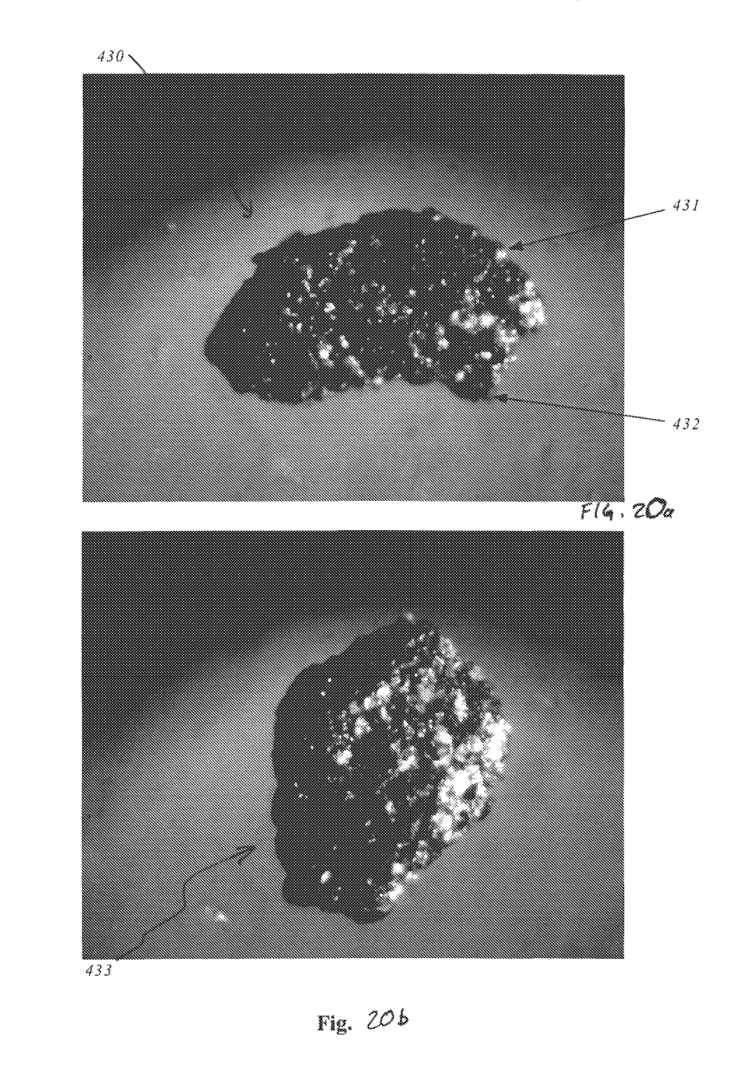

Referring to FIGS. 20a and 20b showing two magnetically-responsive particles corresponding to embodiments described in FIGS. 15a, 15b and 19a-c. An elliptically-shaped magnetically-responsive particle 430 (FIG. 20a) contains hollow glass microspheres 431 a magnetically-responsive material mixed with a matrix material which has been caused to polymerize to form a solid assemblage 432. An alternative example illustrates a teardrop-shaped magnetically-responsive particle 433 (FIG. 20b) of the same materials as magnetically-responsive particle 430.

Reference is made to FIGS. 12a and 12b. In accordance with another embodiment of the invention, an exemplary process for manufacturing magnetically-responsive particles includes:

step 12-1 (FIG. 12b), which is specifying a target final density range for the magnetically-responsive particles;

step 12-2 (FIG. 12b), which is providing particles of a first material 200 (FIG. 12a) which have a density range that is lower than the final target density range;

step 12-3 (FIG. 12b), which is providing a second material 202 (FIG. 12a), which is a coating material 202, which may include an MRM 204 and a matrix material 206, wherein the coating material 202 has a density range that is higher than the final target density range; and

step 12-4 (FIG. 12b), which is covering at least a portion of the surface of the particles of the first material 200 (FIG. 12a) with the coating material 202 to make magnetically-responsive particles having a density range that is generally within the specified (ie. selected) final target density range.

The first material 200 is a core material and may be, for example, hollow glass or ceramic spheres or shapes, hollow or solid polymer spheres or shapes, polystyrene or sawdust. The matrix material 206 may include, for example, one or more of a multi-part-epoxy, cellulose, paper or ceramic. As shown at 208 in FIG. 12a water may be added to dilute the coating material 202 to provide a relatively even coating on the particles of the core material 200 when the particles of first material 200 are added. The coating material 202 bonds to the particles of core material 200 and results in magnetically-responsive particles that are generally within the specified (ie. selected) density range

The magnetically-responsive particles may be dried as necessary in a dryer shown at 210 in FIG. 12a, and at step 12-5 in FIG. 12b and may be sieved as necessary in a classifier 212 in FIG. 12a and at step 12-6 in FIG. 12b to obtain a specified (ie. selected) size and/or density range.

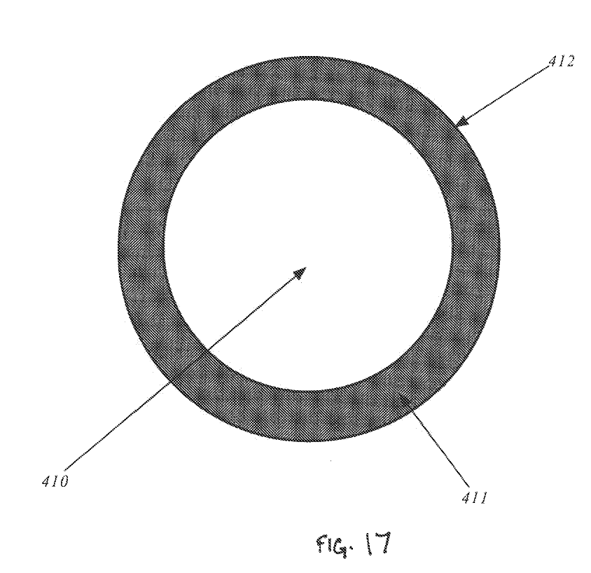

Referring to FIG. 17 illustrating the magnetically-responsive particles produced by the process described by FIGS. 12a and 12b. The magnetically-responsive material that is of a density higher than the target density is mixed with a coating material 411 in FIG. 17 and applied to a base material 410 in FIG. 17 that is of a lower density than target density; the result is a particle 412 of specified (ie. selected) size and density. In alternative embodiments, the magnetically-responsive particles may include, without limitation, other shapes such as ellipsoidal, teardrop or cylinders, such that the various shapes can be used to identify magnetically-responsive particles from different manufacturing batches.

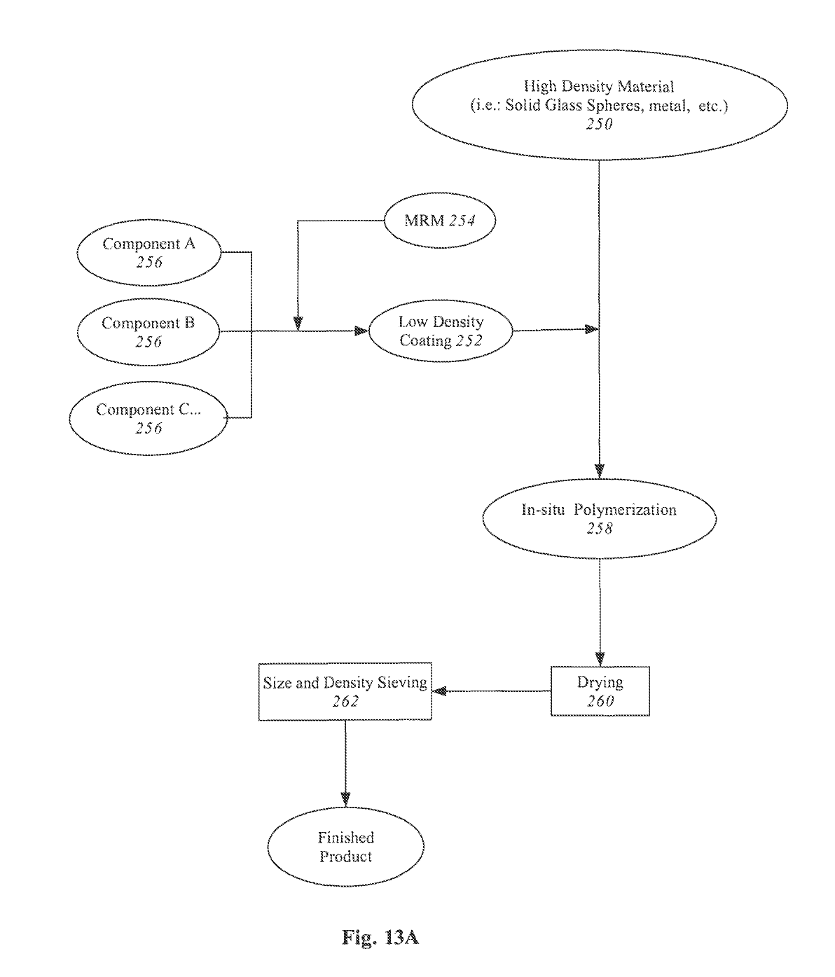

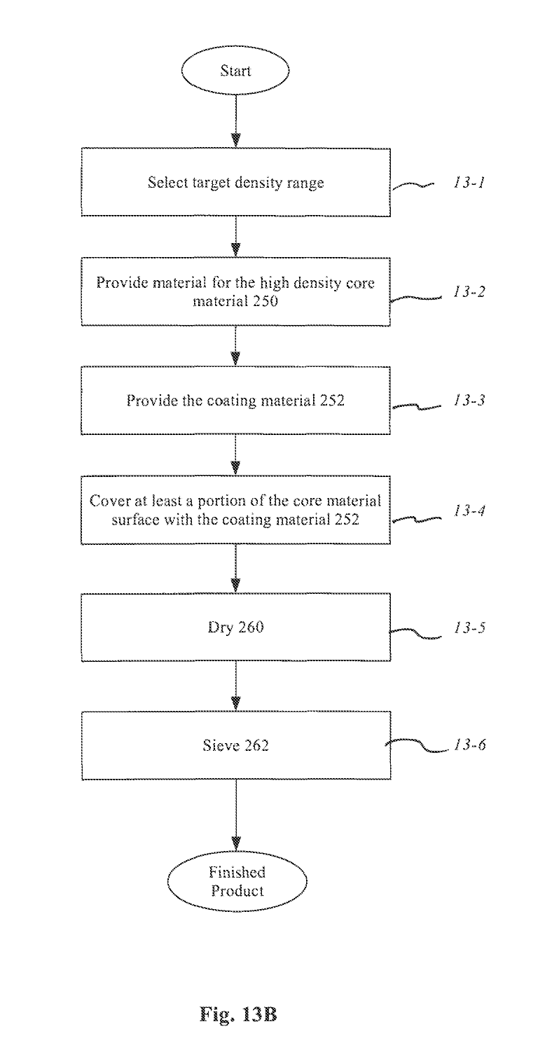

Reference is made to FIGS. 13a and 13b. In accordance with another embodiment of the invention, an exemplary process for manufacturing magnetically-responsive particles includes:

step 13-1 (FIG. 13b), which is specifying a target final density range for the magnetically-responsive particles;

step 13-2 (FIG. 13b), which is providing particles of a first material 250 which have a density range that is higher than the final target density range;

step 13-3 (FIG. 13b), which is providing a second material 252, which is a coating material 252, which may include an MRM 254 and a matrix material 256, wherein the coating material 252 has a density range that is lower than the specified (ie. selected) final target density range; and

step 13-4 (FIG. 13b), which is covering at least a portion of the surface of the particles of the core material 250 with the coating material to make magnetically-responsive particles having a density range that is generally within the specified (ie. selected) final target density range.

The first material 250 may be, for example, solid glass spheres, metal, non-metal, or a polymer. The matrix material 256 may include, for example, one or more of hollow glass spheres, multi-part-epoxy, paper, polymers and sawdust. Water may be added to dilute the coating material 252 so that it forms a relatively even coating on the particles of the core material 250 when the particles of core material 250 are added. The coating material 252 bonds to the particles of core material 250 and results in magnetically-responsive particles that are generally within the specified (ie. selected) density range. The coating material can be applied by in-situ polymerization (shown at 258) or via a multi-part-epoxy.

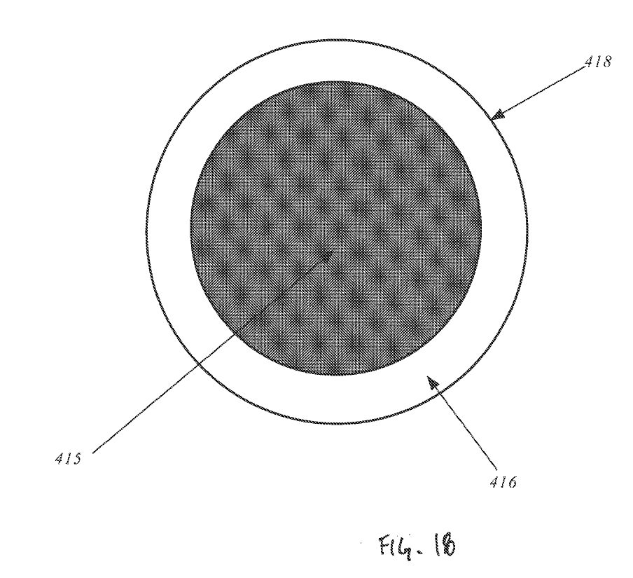

Referring to FIG. 18, illustrating the magnetically-responsive particles produced by the process described by FIGS. 13a and 13b. The magnetically-responsive material is mixed with a coating material 416 in FIG. 18 that is lower than the target density and applied to a base material 415 in FIG. 18 that is of a higher density than target density; the result is a magnetically-responsive particle 418 of specified (ie. selected) size and density. In alternative embodiments, the magnetically-responsive particles may include but are not limited to other shapes such as ellipsoidal, teardrop or cylinders, such that the various shapes can be used to identify magnetically-responsive particles from different manufacturing batches.

The magnetically-responsive particles may be dried as necessary in a dryer 260 in FIG. 13a, and at step 13-5 in FIG. 13b, and may be sieved as necessary in a classifier 262 in FIG. 13a, and at step 13-6 in FIG. 13b to obtain a specified (ie. selected) size and/or density range.

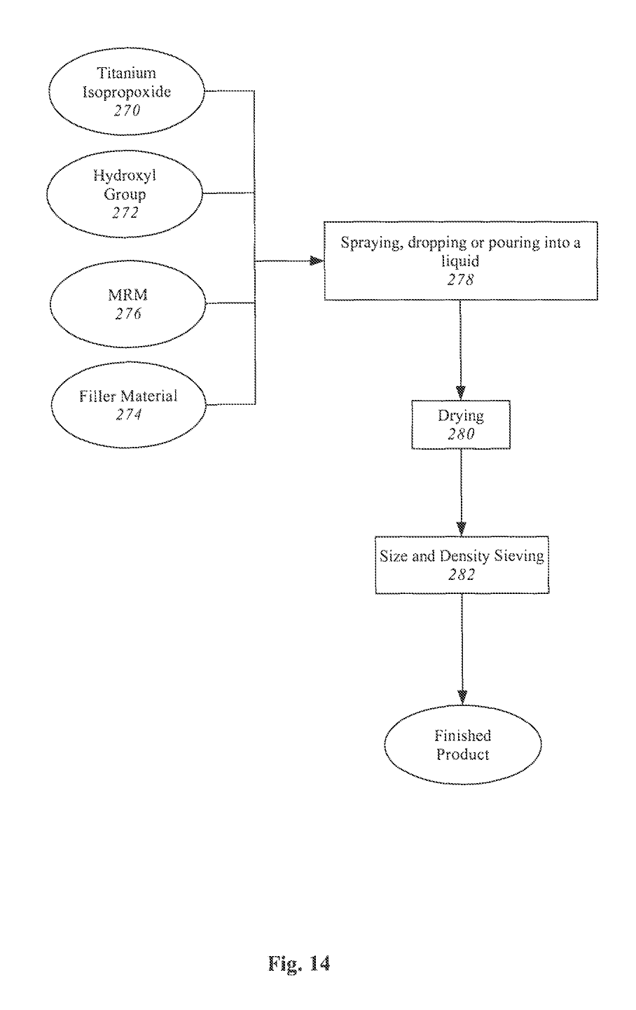

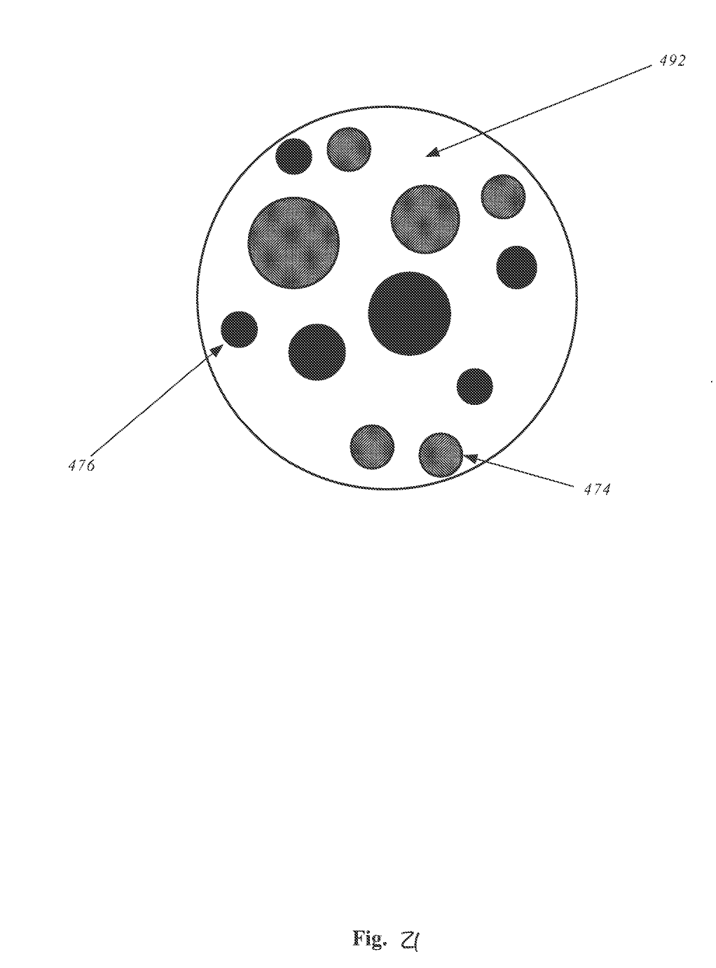

Reference is made to FIG. 14. In accordance with an embodiment of the invention, a specific process for manufacturing magnetically-responsive particles that include in-situ polymerization. For example, a base material, titanium isopropoxide 270 may be provided, which reacts with hydroxyl groups 272 very rapidly. Combining the titanium isopropoxide with a filler material 274, such as a silanol-terminated polydimethylsiloxane, a polymer will form. By adding excess titanium isopropoxide to the system; a chemical equilibrium develops causing the polymer chains to shorten, and the system becomes a liquid. MRM 276 may be added at any suitable time. If the liquid that is formed comes in contact with moist air or water, it hardens and a polymer is formed. Spraying, dropping or pouring this mixture into water in droplets, shown at 278, causes spheres of a specified (ie. selected) density range to form in the water. The spheres may be dried in a dryer 280 as necessary and may be sieved in a classifier 282 as necessary to form the magnetically-responsive illustrated in FIG. 21 showing a particle manufactured by the process above, and illustrating the MRM 476, the filler material 474 and the base material 492 specified (ie. selected)

In another embodiment of the invention, glass spheres in the size range 1-500 microns, either solid or hollow (gas-filled), are mixed with magnetite powder. The mixture is then blended with a liquid such as epoxy resin that solidifies and bonds the materials together. The masses and the volumes of all constituent materials are conserved upon mixing, leading to equations similar to equation (1) that describe the proportions of constituents used to obtain the desired approximate density of magnetically-responsive particles.

In another embodiment of the invention, glass spheres in the size range 1-500 microns, either solid or hollow (gas-filled), are caused to be coated with fine magnetite powder. The thickness of the magnetite coating affects the relative proportions of glass, gas (if any) and magnetite, and hence can be used to control the density of the resulting magnetically-responsive particle. The resulting magnetically-responsive particle is then coated with a protective material to prevent the magnetite from chemically or physically interacting with the surrounding fluid.

In another embodiment the magnetically-responsive particles may be placed in a magnetic field prior to or during their release in a fluid to induce magnetism in the magnetically-responsive particles such that the magnetically-responsive particles behave as permanent or temporary magnets and thus will become attracted to each other to form “flocs”. Controlling the degree of induced magnetism in the magnetically-responsive particles allows the magnetically-responsive particles to simulate the behavior of flocculated particulates with negative, neutral or positive buoyancy and/or to simulate the behavior of a range of materials or particulates with differing degrees of tendency to flocculate.

Additionally or alternatively, in an embodiment of the present invention, a coating of flocculation-control material can be further chosen to control the rate at which the magnetically-responsive particles are attracted to each other to form groups of magnetically-responsive particles called “flocs”. Controlling the rate of attraction between magnetically-responsive particles allows the magnetically-responsive particles to simulate settling behavior in small-grained sediments such as clay or mud.

Referring to FIG. 21 showing the difference between a ‘non-flocing’ 435 and ‘flocing’ 437 magnetically-responsive particles. The size of the ‘flocs’ 439 can be controlled by the amount and type of coating applied to the individual particles along with the size and distribution of the magnetically-responsive particle size. Alternatively, the ‘floc’ size can be controlled by the level of magnetism that the magnetically-responsive particles posses, the greater the magnetism and larger the ‘flocs’ the lower the magnetism the smaller the ‘flocs’.

In embodiments wherein a flocculation control material is used, it may be selected to influence the mutual attraction of the magnetically-responsive particles, while retaining the magnetically-responsive properties necessary to be collected by magnetic particle-collectors.

Generally, aspects of the present invention include the manufacture of magnetically-responsive particles using any mixture of materials necessary to produce magnetically-responsive particles having characteristic distribution ranges, narrow or broad, for specified (ie. selected) specific gravity, buoyancy, size, rise or sinking rate, shape and color around a mean or mode or median for each characteristic. Somewhat more specifically, in accordance with some aspects of the present invention the manufacturing process includes steps by which magnetically-responsive particles are produced with a specified (ie. selected) size, measured as equivalent spherical diameter, with a specified (ie. selected) (narrow or broad) distribution of sizes around a specific mean. Somewhat more specifically, in accordance with some aspects of the present invention the manufacturing process includes steps by which magnetically-responsive particles are produced with a specified (ie. selected) color or range or mixture of colors, fluorescent or otherwise. Somewhat more specifically, in accordance with some aspects of the present invention the manufacturing process includes steps by which magnetically-responsive particles are produced with a specified (ie. selected) shape or range of shapes, perfect or imperfect, including irregular, spherical, ovoid, plate, filament, or polyhedral etc. Somewhat more specifically, in accordance with some aspects of the present invention the manufacturing process includes steps by which magnetically-responsive particles are produced such that they are resistant to any form of bio-fouling. Somewhat more specifically, in accordance with some aspects of the present invention the manufacturing process includes steps by which magnetically-responsive particles are produced such that they are biodegradable within a specified (ie. selected) period (short or long) with a distribution of degradation time around a specific mean.

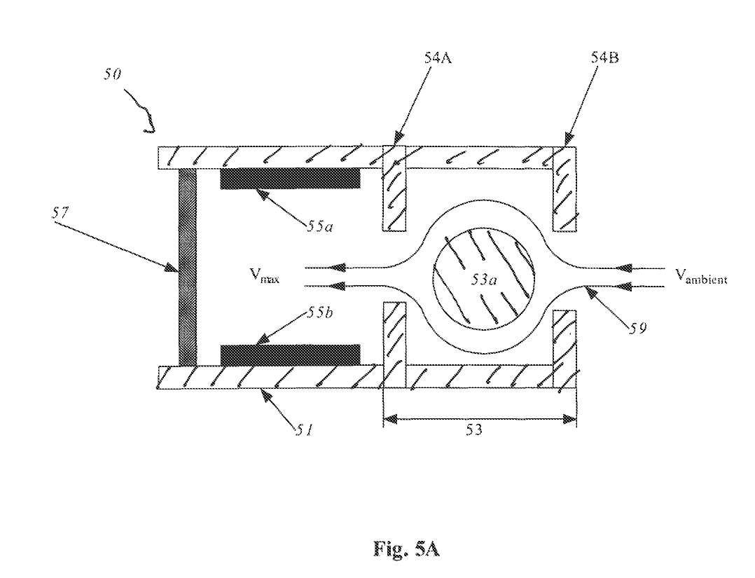

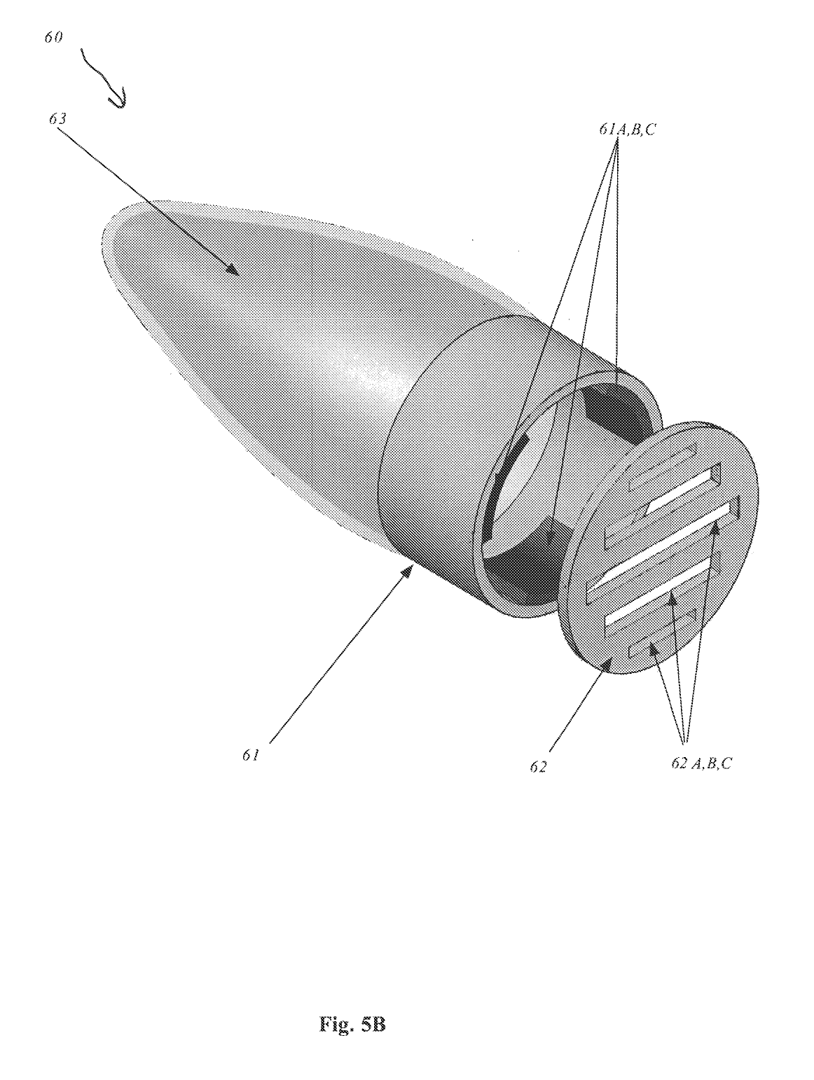

Referring now to FIGS. 5a and 5b, shown are two examples of magnetic particle-collectors according to embodiments of the invention.

Specifically, shown in FIG. 5a is a cross-sectional view of a magnetic particle-collector 50 in accordance with an embodiment of the present invention. The first magnetic particle-collector 50 includes a tubular body 51 with first and second open ends 54a and 54b and which defines a fluid flow path shown at 59. The first end 54b is the inlet end and optionally includes a flow controller 53 to control the flow of fluid through the magnetic particle-collector 50. The flow controller 53 may be a ball valve, as shown at 53a in FIG. 5a. As such, the position of the valve element, (i.e. the ball) may be adjusted to control the velocity of the fluid flow through the tubular body 51. Instead of a ball valve, 20 it is alternatively possible for another type of flow controller 53 to be used, such as some other kind of valve or such as an orifice plate.

The second end of the magnetic particle-collector 50 is the outlet end and is optionally covered by a fluid permeable mesh element 57, such as a screen, that allows fluid to flow through, but is impermeable to magnetically-responsive particles. Inside the tubular body 51, the first magnetic particle-collector 50 includes magnetic elements 55a and 55b, such as permanent magnets or electromagnets. In this particular embodiment the magnetic elements 55a and 55b are fixed to the inside wall of the tubular body 51. However, in alternative embodiments, the magnetic elements 55a and 55b may be positioned to exert a magnetic force on at least a portion of the magnetically-responsive particles in the fluid flow path 59. The magnetic elements 55a and 55b may achieve the aforementioned positioning by any suitable means. For example, the magnetic elements 55a and 55b may be tethered to the body 51 in the fluid flow path 59. Alternatively, the magnetic elements 55a and 55b may be freely placed inside the tubular body. In the latter case it is preferable that the magnetic elements are prevented from falling free of the first magnetic particle-collector 50 (e.g. by the shape of the tubular body 51). In alternative embodiments, the first magnetic particle-collector 50 does not include the screen 57. Alternatively, the magnetic elements 55a and 55b may be themselves positioned outside of the fluid flow path 59 but may be proximate to the flow path 59 so that they at exert a magnetic force on magnetically-responsive particles in the flow path 59 while not themselves being exposed to the flow. In such an embodiment, the magnetic elements 55a and 55b may be, for example, positioned just behind the wall of the tubular body 51.

In operation, the fluid, possibly carrying magnetically-responsive particles, flow through the ball valve 53a into and out of the tubular body 51. When fluid flows in, carrying magnetically-responsive particles, the magnetically-responsive particles are attracted to and retained on the magnetic elements 55a and 55b, which inhibits them from flowing back out through the ball valve 53a. Fluid can flow out of the tubular body 51 at the outlet end, via the optional mesh element 57, which is impermeable to the particles. It is also possible for fluid to flow out of the collector 50 via the ball valve 53a.

The ball valve 53a controls the maximum velocity Vₘₐₓ of the fluid through the magnetic particle-collector 50. Ambient fluid flow has a velocity of V_{ambient}. Vₘₐₓ may be selected to be greater than, less than, or the same as V_{ambient}, In embodiments where the maximum fluid velocity Vₘₐₓ is selected to be less than the ambient fluid velocity V_{ambient}, the ball valve 53a provides a selected restriction in the flow path 59.

Turning to FIG. 5b, provided is a perspective view of another magnetic particle-collector 60 according to an embodiment of the invention. The magnetic particle-collector 60 includes a body 61, which may also be referred to as a collar 61 that holds magnetic elements 61a, 61b and 61c, an optional mesh element 63, such as a generally cone-shaped net, that is impermeable to magnetically-responsive particles, and an optional cover 62. The body 61 defines a flow path for fluid carrying magnetically-responsive particles. The body 61 may be rigid or semi-rigid. The body 61 may be tubular or may have some other suitable configuration.

The magnetic elements 61a, 61b and 61c may be positioned on the inside wall of the rigid tubular collar 61. While three magnetic elements 61a, 16b and 61c are shown in the exemplary structure shown in FIG. 5b, it will be appreciated that other numbers of magnetic elements may be provided.

The optional cover 62 fits at one end of the body 61 and includes slots 62a, b, c. The slots 62a, b, c serve to control the velocity of fluid into the collector 60 and thus act as flow controllers. The cover 62 may be removable to facilitate access by a person to the magnetically-responsive particles collected in the magnetic particle-collector 60.

The mesh element 63 is shown as being generally cone-shaped; however it may alternatively have any other suitable shape. In alternative embodiments the cone-shaped net 63 is open-ended to allow the fluid to flow though the magnetic particle-collector while acting to vane the magnetic particle-collector into the fluid-flow.

In operation, the fluid, possibly carrying magnetically-responsive particles, flow through the slots 62a, b, c through the collar 61 and out through the cone-shaped net 63. When fluid flows in, carrying magnetically-responsive particles, the particles are attracted to the magnetic elements 61a, b and c, which inhibit the magnetically-responsive particles from flowing back out through the slots 62a, b, c. Fluid can flow out the cone-shaped net 63, which is impermeable to the magnetically-responsive particles. It is possible that some fluid may flow out of the magnetic particle-collector 60 through the slots 62a, b, c.

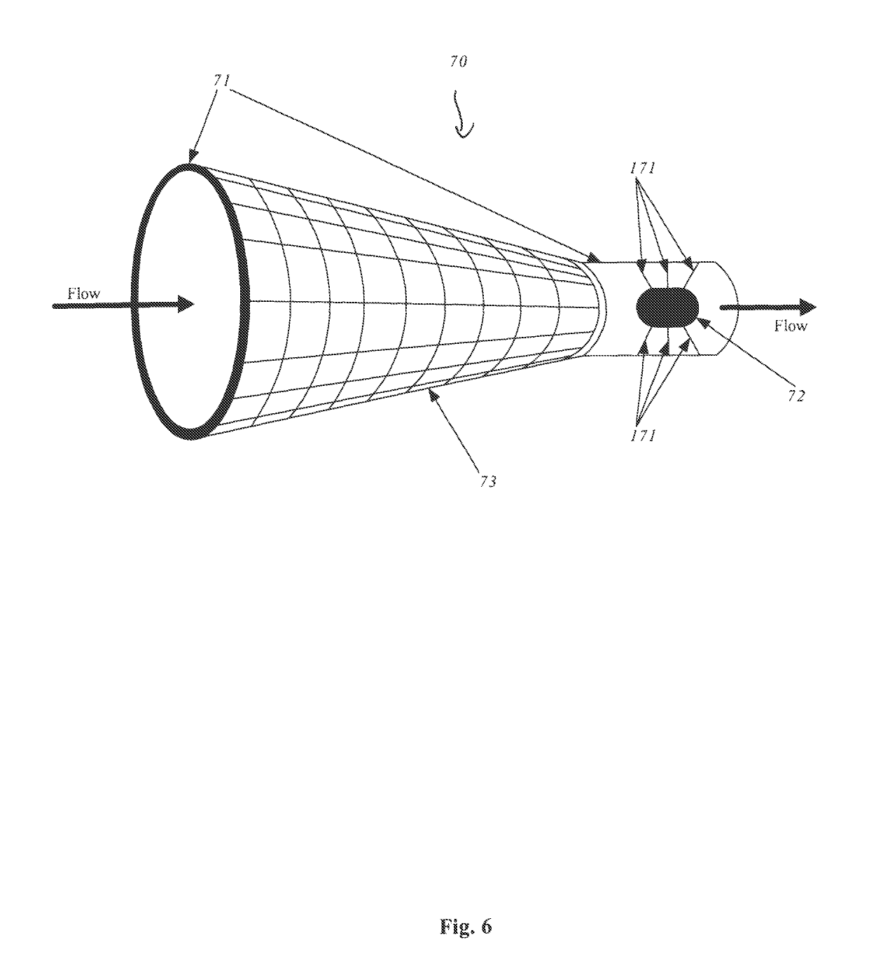

Referring to FIG. 6, provided is a perspective view of another magnetic particle-collector 70 according to an embodiment of the invention. The magnetic particle-collector 70 includes a body 71 which may also be referred to as a collar 71, a magnetic element 72 and a concentrating net 73.

The magnetic element 72 is shown as being suspended in the flow path defined by the body 71, by a set of tethers 171. The magnetic element 72 could alternatively be mounted to the body 71 in any other suitable way, such as, for example, about the inner wall of the body 71, similar to the embodiments shown in FIGS. 5a and 5b.

The concentrating net 73 is positioned at the inlet end of the body 71. The concentrating net 73 is permeable to the fluid but is impermeable to the magnetically-responsive particles in the fluid flow. Also the concentrating net 73 has a diameter that generally decreases in a downstream direction. As fluid flows along the net 73, some fluid is permitted to escape through the net 73, thereby increasing the concentration of whatever magnetically-responsive particles are contained in the fluid remaining

In operation, the fluid, possibly carrying magnetically-responsive particles, flows through the net 73 and the magnetically-responsive particles are guided to and through the body 71. When fluid flows in, carrying magnetically-responsive particles, the magnetically-responsive particles are attracted to the suspended magnetic element 72 that traps and collects the magnetically-responsive particles by means of its magnetic field.

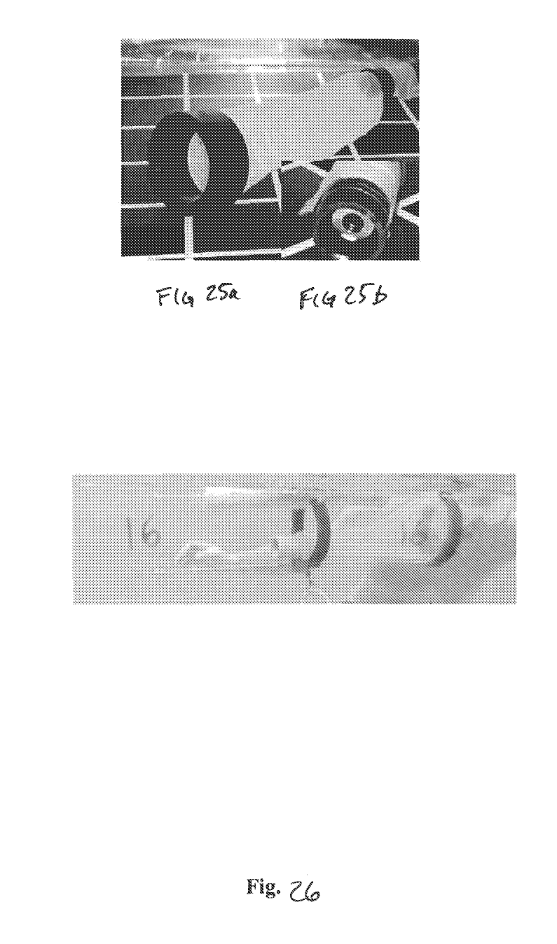

Referring to FIGS. 25a, 25b and 26 are shown photographic images of a partially disassembled and an in-situ moored magnetic particle-collector as schematically illustrated in FIG. 6.

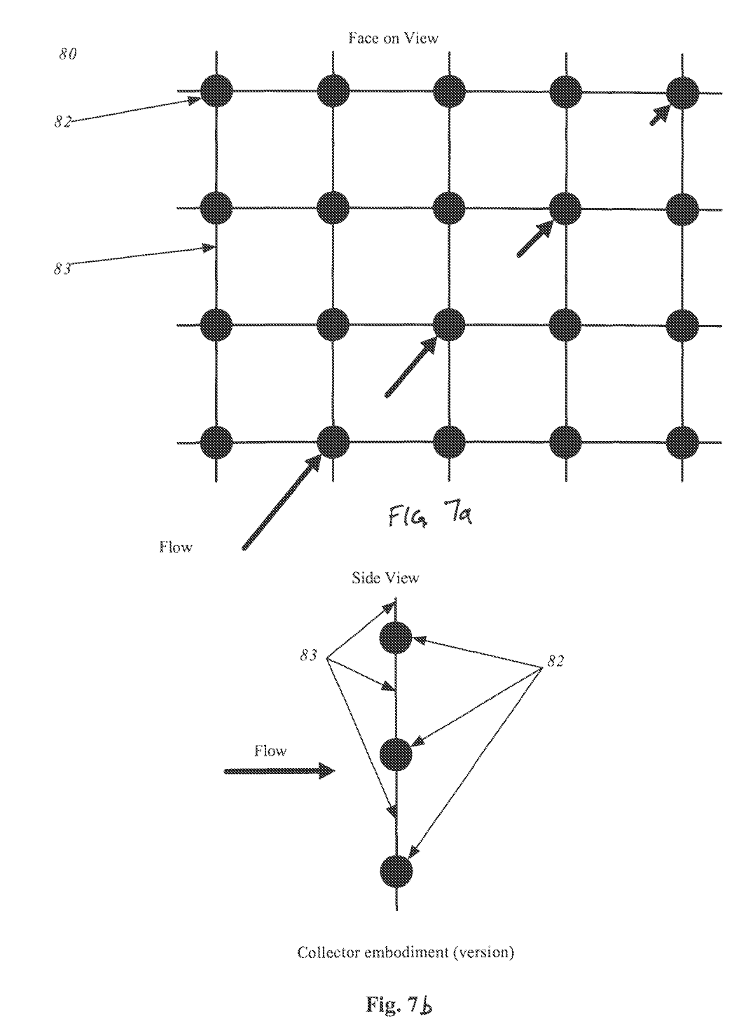

Referring to FIGS. 7a and 7b, provided are magnified views of a portion of a magnetic particle-collector grid 80 according to an embodiment of the invention. The particle-collector grid 80, shown in face view in FIG. 7a and side view in FIG. 7b, includes a number of magnetic elements 82 interconnected to form a grid pattern by a body that is made up of flexible (or rigid) connecting elements 83.

In operation, the fluid, possibly carrying magnetically-responsive particles, flows through and/or along the magnetic particle-collector grid 80. The magnetically-responsive particles are attracted to the suspended magnetic elements 82, which trap and collect the magnetically-responsive particles by means of their respective magnetic fields.

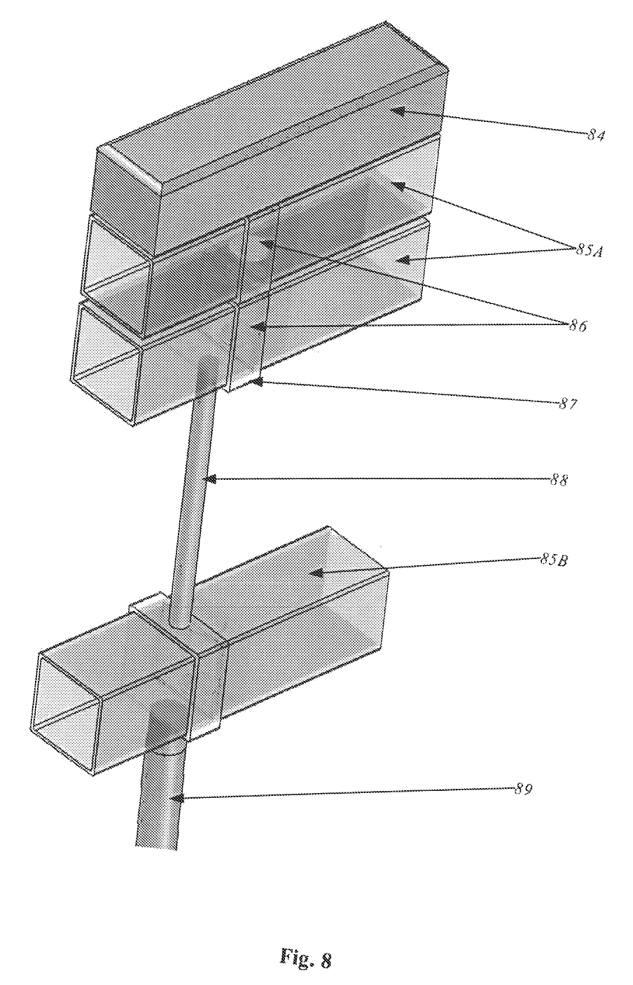

Referring to FIG. 8 is a transparent view of a magnetic particle-collector array 180 according to an embodiment of the invention. The magnetic particle-collector array 180 comprises an optional float 84 made of a material with low density (i.e. a density that is lower than that of the surrounding fluid) so as to support the magnetic particle-collector array 180 at or near the surface of the fluid, one or more top magnetic particle-collectors 85a suspended at or near the fluid surface, magnetic elements 86 suspended within magnet housings 87 so as to allow convenient removal for examination, a spacer 88, and one or more lower magnetic particle-collectors 85b that also contain one or more magnetic elements 86 within magnet housings 87. The magnetic particle-collector array 180 can optionally be maintained at a desired location by means of attachment to a mooring line 89 that is held in place by an anchor (not shown), or alternatively be allowed to drift within the fluid being monitored or to be suspended from the surface.

In operation, the fluid, possibly containing magnetically-responsive particles flows through the magnetic particle-collector collector array 180 and near the magnetic elements 86. The magnetically-responsive particles are attracted to the magnetic elements 86, which trap and collect the magnetically-responsive particles. The magnetic particle-collectors 85a and 85b are suspended at varying depths at and/or below the surface of the fluid to collect the magnetically-responsive particles at the varying depths. It will be understood that the magnetic particle-collector array 180 could optionally include magnetic particle-collectors at more than two heights. There could, for example, be one or more upper magnetic particle-collectors, one or more intermediate magnetic particle-collectors and one or more lower magnetic particle-collectors. It will also be understood that the magnetic particle-collector array 180 could optionally have as little as a single magnetic particle-collector at a single height. In such an instance, the magnetic particle-collector array 180 would instead be referred to as a magnetic particle-collector 180. The magnetic particle-collector 180 would not require the spacer 88.

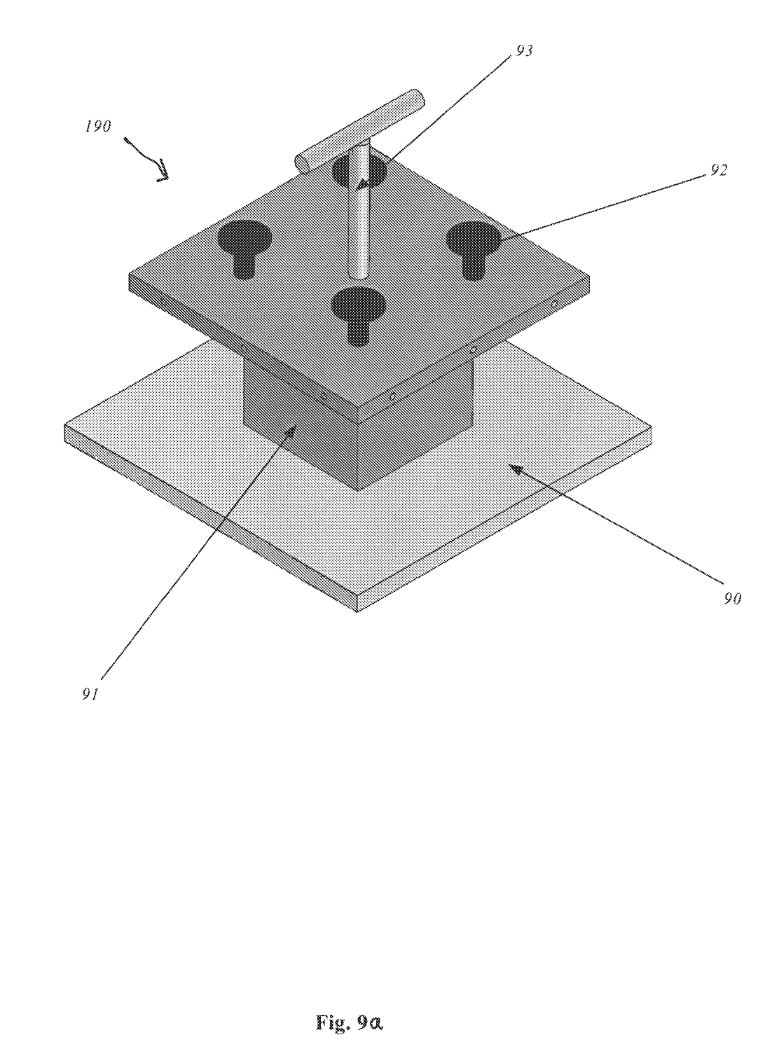

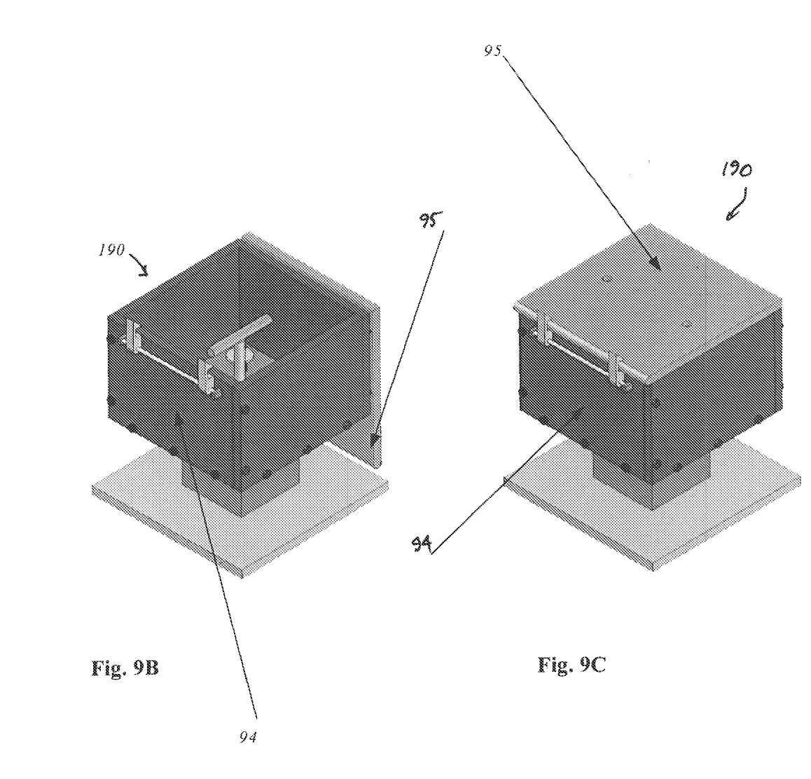

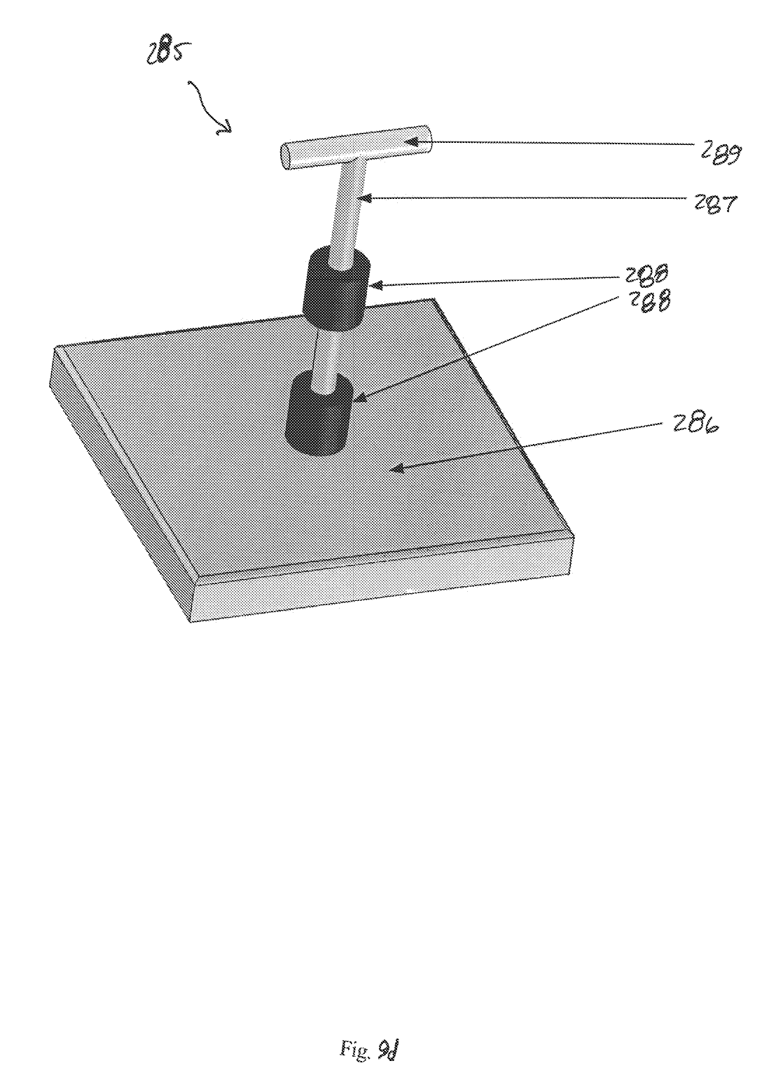

Referring to FIG. 9a, provided is a view of the internal components of a magnetic particle-collector 190 designed to rest at the bottom of a while attracting magnetically-responsive particles that have a greater density than the fluid and thus settle-out of the fluid flow. The magnetic particle-collector 190 comprises a base 90 that supports a generally vertical stand 91 upon which are mounted one or more magnetic elements 92 a selected vertical distances above the base 90. At the top of the stand 91 may be a handle 93 used to place and pick up the collector 190. The shaft and base may be made from a material that is substantially magnetically unresponsive, such as stainless steel.

Referring to FIGS. 9b and 9c is a view of the magnetic particle-collector 190, with an optional box 94 that surrounds the magnetic elements 92 and which has a lid 95 that can be opened. In operation, the magnetic particle collector 190 is caused to rest at the bottom of the fluid and the lid 95 is opened, exposing the magnetic elements 92 to the ambient fluid possibly containing magnetically-responsive particles, and will trap and collect the magnetically-responsive particles that settle from the fluid. As illustrated in FIG. 9c, prior to removing the collector 190 from the bottom, the lid 95 is closed, thereby protecting the magnetic elements 92 from disturbance and possible loss of magnetically-responsive particles. It will be understood that the collector 190 may have as few as one magnetic element thereon.

Reference is made to FIG. 9d, which shows a particle collector 285 that may be similar to the particle collector 190 shown in FIGS. 9a, 9b and 9c, but has a simplified construction. The particle collector 285 has a base 286, a vertically extending shaft 287, one or more magnetic elements 288 on the shaft 287 at selected heights, and a handle 289.

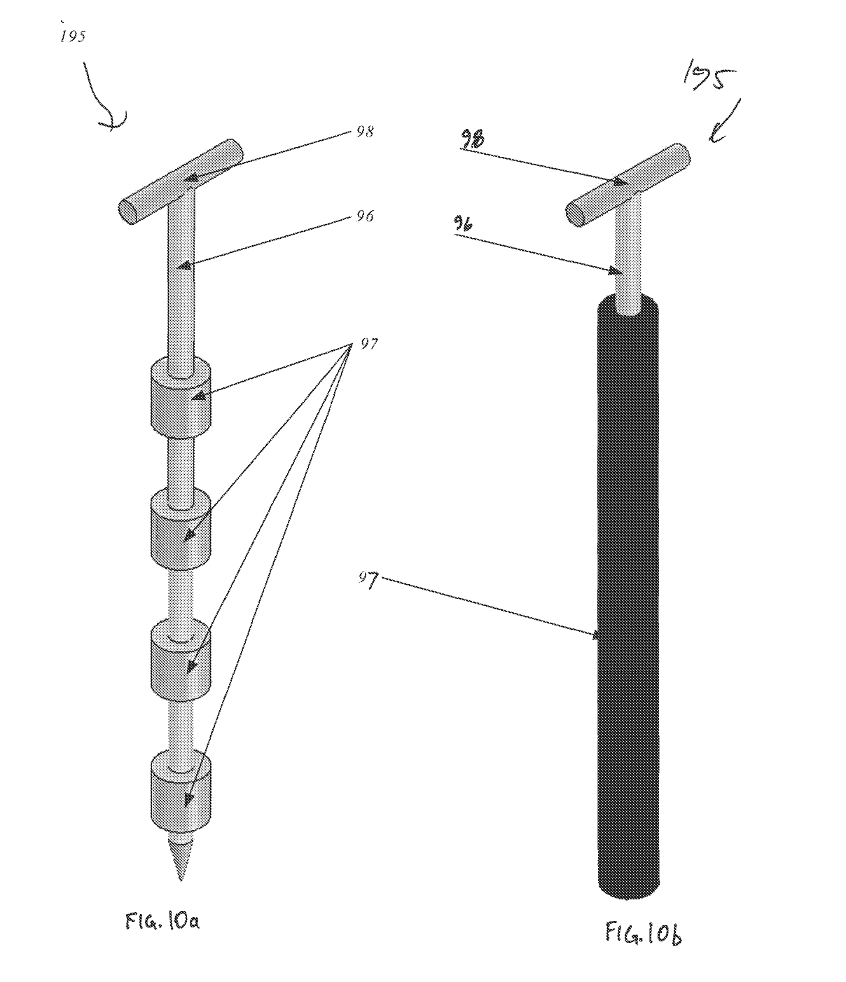

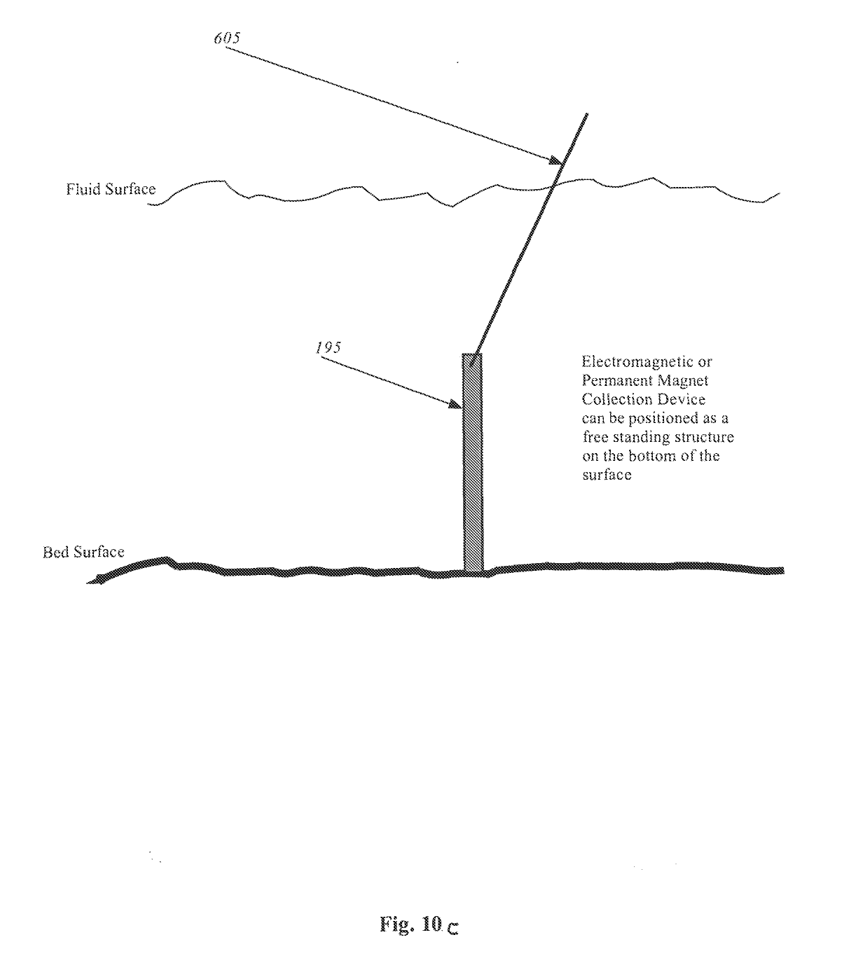

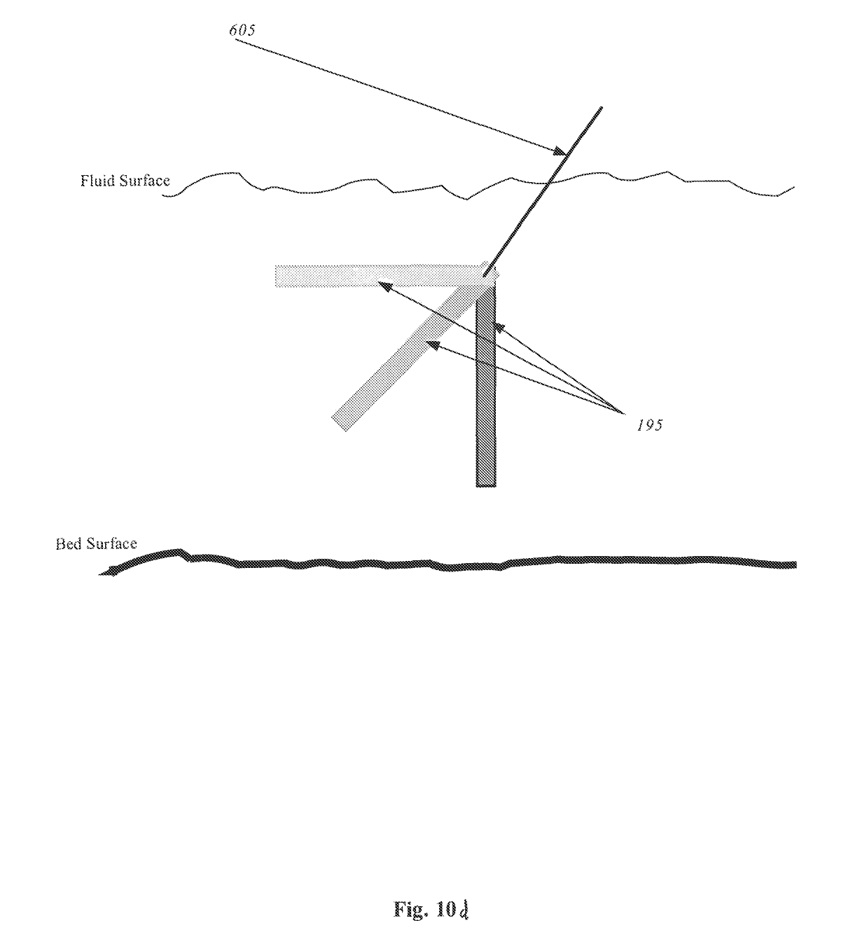

Referring to FIGS. 10a and 10b, provided are views of a magnetic particle-collector 195 according to another embodiment of the invention. The magnetic particle-collector 195 comprises a shaft 96 upon which are mounted one or more permanent magnetic elements 97 at selected positions along the shaft 96 or one or more electromagnets that can be turned on or off at will. The shaft 96 may be made from any suitable material, such as stainless steel. In one configuration, the top of the shaft 96 may have a handle 98 that is used to insert the shaft 96 partially or fully into the sediments at the bottom of the fluid, and is also used to recover the magnetic particle-collector 195.

Referring to FIGS. 10c and 10d, provided are views of other configurations, in operation, of the magnetic particle-collector 195 that may be free-standing, free-drifting or suspended in the fluid or is towed either vertically, horizontally or at any selected angle in the fluid by a line or platform 605. In certain operations the magnetic particle-collector 195 is left free-standing in the fluid for a selected period (FIG. 10c), or is towed at a selected angle (FIG. 10d) through the fluid for a selected time and/or distance. Magnetically-responsive particles in the fluid that flow near the magnetic particle-collector magnetic elements will be attracted to the magnetic elements which trap and collect the magnetically-responsive particles. Upon recovery, the number of magnetically-responsive particles collected can be quantified and this can be achieved periodically at intervals over a sample period or sample distance to collect more information about the fluid system.

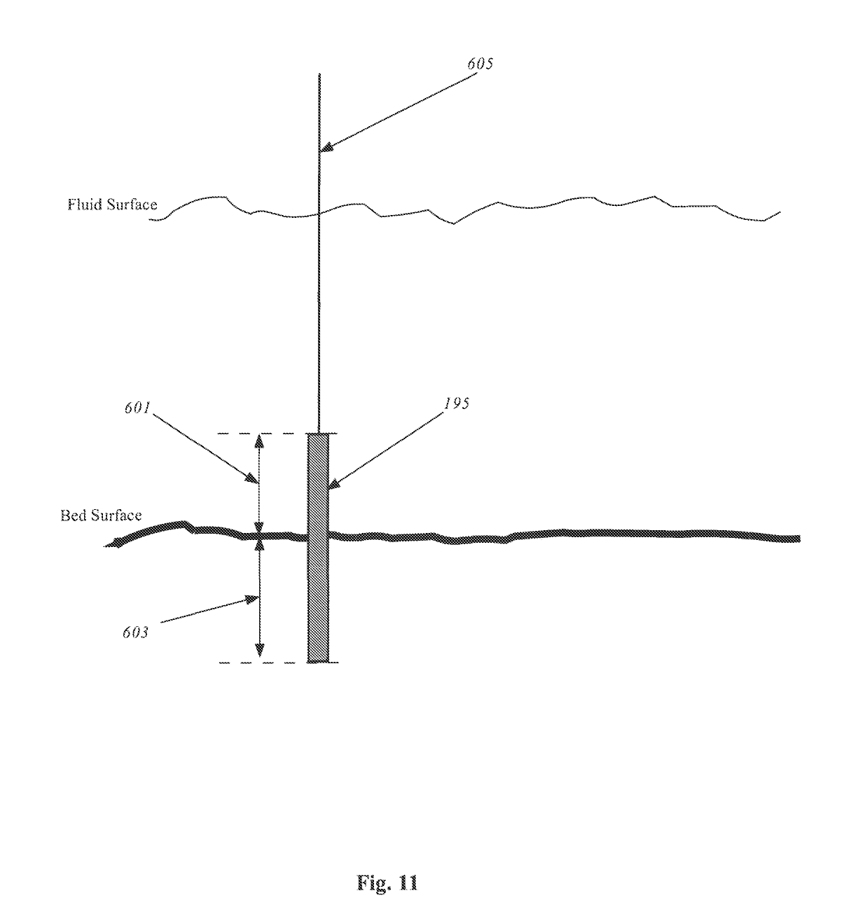

With reference to FIG. 11, in certain operations the magnetic particle-collector 195 is inserted partially or fully into the sediments to the desired depth 603 and magnetically-responsive particles in the sediments, and near the magnetic particle-collector's magnetic elements 97 in FIG. 10 when 195 is inserted into the sediments, will be attracted to the magnetic elements that trap and collect the magnetically-attractive particles above 601 and within 603 the sediments. Prior to recovery, a cover is placed over the magnetic particle-collector 195 and sealed to protect the magnetic elements and magnetically-responsive particles from disturbance and possible loss of magnetically-responsive particles during recovery.

While the above description provides example embodiments, it will be appreciated that the present invention is susceptible to modification and change without departing from the fair meaning and scope of the accompanying claims. Accordingly, what has been described is merely illustrative of the application of aspects of embodiments of the invention. Numerous modifications and variations of the present invention are possible in light of the above teachings. It is therefore to be understood that within the scope of the appended claims, the invention may be practiced otherwise than as specifically described herein.

We claim:

1. A magnetically-responsive particle, for use in a fluid with a first density comprising:
a base material with a second density that is lower than the first density of the fluid; and
a magnetically-responsive material (MRM) with a third density that is higher than the density of the first density of the fluid;
wherein the base material and the magnetically-responsive material are combined in a ratio to provide the magnetically-responsive particle with a fourth density that is selected based on the first density.

2. A magnetically-responsive particle according to claim 1, wherein the size of the particle is in the range of 5 microns-20 mm.

3. A magnetically-responsive particle according to claim 1, wherein the base material includes a dye.

4. A magnetically-responsive particle according to claim 1, wherein the base material includes at least one type of natural fibre.

5. A magnetically-responsive particle according to claim 1, wherein the base material includes a particle of a core material and a matrix material, wherein the MRM and matrix material form a coating on the particle of the core material.

6. A magnetically-responsive particle according to claim 5, wherein the particle of the core material is a hollow glass sphere that has a density that is lower than the first density.

7. A magnetically-responsive particle according to claim 5, wherein the particle of the core material is a solid glass sphere that has a density that is higher than the first density.

8. A magnetically-responsive particle according to claim 5, wherein the particle of the core material is a solid glass sphere that has a density that is higher than the first density.

9. A magnetically-responsive particle according to claim 1, further comprising a protective coating that is external to the magnetically-responsive material, wherein the coating inhibits interaction between the fluid and the magnetically-responsive material.

10. A particle collector for collecting magnetically-responsive particles in a fluid, comprising:
a body defining a fluid flow path; and
at least one magnetic element positioned to exert an attractive magnetic force on magnetically-responsive particles in the fluid flow path, such that the at least one magnetic element retains the magnetically-responsive particles.

11. A particle collector according to claim 10, further comprising a flow controller to control the velocity of a fluid within the body.

12. A particle collector according to claim 11, wherein the flow controller obstructs flow of fluid in the fluid flow path.

13. A particle collector according to claim 12, wherein the flow controller obstructs flow of fluid in the fluid flow path by an adjustable amount.

14. A particle collector according to claim 10, further comprising a mesh element that is positioned upstream from the body, wherein the screen is generally tubular and has an inlet end and an outlet end, wherein the inlet end has a greater cross-sectional area than the outlet end, wherein the screen is permeable to fluid flow there-through, but is impermeable to the flow of magnetically-responsive particles there-through.

15. A particle collector according to claim 10, wherein the body comprises a plurality of interconnecting elements and wherein the interconnecting body elements and magnetic elements are arranged to form a grid through which fluid can flow.

16. A particle collector according to claim 15, wherein the magnetic elements are positioned at intersection points on the grid.

17. A particle collector for collecting magnetically-responsive particles in a fluid, comprising:
a shaft; and
at least one magnetic element connected to the shaft, wherein the magnetic element is positioned to exert an attractive magnetic force on magnetically-responsive particles in fluid passing by the shaft, such that the at least one magnetic element retains the magnetically-responsive particles.

18. A particle collector according to claim 17, wherein the at least one magnetic element has a height and is positioned to collect magnetically-responsive particles flowing at a selected height range in the fluid.

19. A particle collector according to claim 17, wherein the particle collector includes a plurality of magnetic elements positioned at selected heights on the shaft to collect magnetically-responsive particles flowing at a plurality of selected height ranges in the fluid.

20. A particle collector according to claim 17, further comprising a float connected to the rest of the particle collector for positioning the particle collector at a selected height relative to the surface of the fluid.

21. A particle collector according to claim 17, further comprising a base connected to the bottom of the shaft, for resting the particle collector on a bed surface at the bottom of the fluid.

22. A particle collector according to claim 21, further comprising a housing, wherein the housing includes a housing wall that is positioned around the at least one magnetic element wherein the housing includes a lid, wherein the lid is positionable in an open position to permit fluid flow to the at least one magnetic element, and a closed position to restrict fluid flow to the at least one magnetic element to inhibit disturbance of magnetic particles collected on the at least one magnetic element during removal of the particle collector from the fluid.

23. A particle collector according to claim 17, further comprising a housing, wherein the shaft has a tip that is shaped to facilitate penetration of the shaft into a bed surface at the bottom of the fluid.

24. A particle collector according to claim 23, wherein at least one of the at least one magnetic element is positioned proximate the tip so that during use, the at least one of the at least one magnetic element is at least partially beneath the bed surface.

25. A particle collector array including a plurality of particle collectors as claimed in claim 10, wherein the particle collectors are positioned at selected heights in the array for collecting particles at a plurality of selected heights in the fluid.

26. A particle collector array as claimed in claim 25, further comprising a float connected to the rest of the particle collector array for positioning the particle collectors at selected heights relative to the surface of the fluid.

27. A system for evaluating a fluid system, comprising:
a batch of magnetically-responsive particles; and
at least one particle collector, wherein the particle collector includes a body and at least one magnetic element, wherein the body defines a fluid flow path, and wherein the at least one magnetic element is positioned to exert an attractive magnetic force on at least some fraction of any magnetically-responsive particles in the fluid flow path, such that the at least one magnetic element retains at least some of the magnetically-responsive particles.

28. A method of manufacturing magnetically-responsive particles for use in a fluid comprising:
combining a first amount of a base material with a second amount of a magnetically-responsive material to create a magnetically-responsive composition, wherein the first and second amounts are selected from a mass ratio derived from the target density of the composition, wherein the target density of the composition is selected based on the density of the fluid; and
forming the magnetically-responsive particles.

29. A method according to claim 28, further comprising pre-drying the first amount of magnetically-responsive material.

30. A method according to claim 28, further comprising heating the magnetically-responsive composition to melt portions of the base material.

31. A method according to claim 30, further comprising extruding and pelletising the heated magnetically-responsive composition to form the magnetically-responsive particles.

32. A method according to claim 30, further comprising solidifying the heated magnetically-responsive composition.

33. A method according to claim 32, further comprising one of size-reducing the solidified magnetically-responsive composition to form the magnetically-responsive particles.

34. A method according to claim 28, further comprising drying the magnetically-responsive particles.

35. A method according to claim 34, further comprising re-processing the magnetically-responsive particles to make smaller magnetically-responsive particles.

36. A method according to claim 28, further comprising adding a surfactant to the magnetically-responsive particles.

37. A method according to claim 28, further comprising magnetizing the magnetically-responsive particles to induce a selected degree of magnetic particle-to-particle attraction so that the particles floc to a selected degree when in a target fluid environment.

38. A method according to claim 37, further comprising coating the magnetically-responsive particles to control the degree to which the particles adhere to each other from magnetic attraction.

39. A batch of magnetically-responsive particles according to claim 1, the batch comprising a mixture of particles of different densities wherein some of the magnetically-responsive particles are negatively buoyant in the fluid, some of the magnetically-responsive particles are neutrally buoyant in the fluid and some of the magnetically-responsive particles are positively buoyant in the fluid.

40. A batch of magnetically-responsive particles according to claim 39, wherein the batch further comprises a mixture of magnetically-responsive particles of different sizes.

41. A batch of magnetically-responsive particles according to claim 39, the batch comprising a mixture of particles of different densities wherein some of the particles are negatively buoyant in the fluid, some of the particles are neutrally buoyant in the fluid and some of the particles are positively buoyant in the fluid, wherein the mixture is bound by a liquid that solidifies so as to create magnetically-responsive particles comprised of the mixture.

42. A batch of magnetically-responsive particles according to claim 39, wherein the magnetically-responsive particles have a selected degree of magnetic attraction to one another so that the particles floc by a selected amount when in a target fluid environment.

43. A batch of magnetically-responsive particles according to claim 42, wherein the particles include a coating that controls the degree to which the particles adhere to each other from magnetic attraction.

44. A particle collector for collecting magnetically-responsive particles in a fluid, comprising:
at least one magnetic element positioned to exert an attractive magnetic force on magnetically-responsive particles in the fluid flow path, such that at least one magnetic element captures the magnetically-responsive particles.
